(19) United States
(12) Patent Application Publication
(10) Pub. No.: US 2021/0343388 A1
(43) Pub. Date: Nov. 4, 2021

(54) Dosage Determination Apparatus

(71) Applicant: Digital Hospital, Inc., San Jose, CA (US)

(72) Inventors: Kevin R Brown, Rescue, CA (US); Philip Curtis, San Jose, CA (US)

(73) Assignee: Digital Hospital, Inc., San Jose, CA (US)

(21) Appl. No.: 17/338,662

(22) Filed: Jun. 3, 2021

Related U.S. Application Data
(63) Continuation-in-part of application No. 15/829,744, filed on Dec. 1, 2017.
(60) Provisional application No. 63/034,379, filed on Jun. 3, 2020.

(51) Int. Cl. G16H 20/17; G16H 10/60; G16H 40/67

(52) U.S. Cl. CPC G16H 20/17; G16H 40/67; G16H 10/60

(57) ABSTRACT

An apparatus for use with electronic health records of a patient to provide a medicament dose instruction for the patient, including a processor configured to search the electronic health records for a medication order of the patient pertaining to the medicament. The processor is configured to search the electronic health records for the at least one medical condition of the patient referenced in the medication order and to determine the prescribed medicament dose as a function of the at least one medical condition of the patient and output the prescribed medicament dose for filling.

### CROSS REFERENCE TO RELATED APPLICATION

This application claims the benefit of U.S. provisional application Ser. No. 63/034,379 filed Jun. 3, 2020 and claims priority to U.S. nonprovisional application Ser. No. 15/829,744 filed Dec. 1, 2017, which claims priority to International Application Number PCT/US2016/035082 filed May 31, 2016, which claims the benefit of U.S. provisional application Ser. No. 62/169,484 filed Jun. 1, 2015, the entire content of each of which is incorporated herein by this reference.

### FIELD OF THE INVENTION

The present invention relates to the measurement of liquids in containers, and more particularly to the measurement of liquid in containers using digital sensors.

### BACKGROUND

Human practices, including the practices of medicine, manufacturing, and science can include adverse events. For example, differing doses of medications which change according to various other current patient parameters can be complicated to transform into a discrete amount that can be measured. A human error transforming a narrative conditional dosage amount into an incorrect discrete dosage amount may be dangerous or fatal to the patient. If the dose deciphered is correct, it can be advisable to confirm that the amount of the dose in the applicator is accurate before administration. Further, the sciences of glucometrics and insulinometrics are both dependent on the knowledge of the dosage of insulin and the type of insulin administered to the hospital patient.

Medical adverse events can result in undesired harmful effects on patients. Reducing such medical adverse events would be desirable. Further, the applied practice of medicine needs a quicker, more accurate, and less error-prone way to measure and enter into the Electronic Health Record (EHR) conditional amounts of medicament before the medicament is prepared as well as before the medicament is given to the patient.

In addition, apparatus and equipment have been provided for determining the characteristics of liquid in containers, for example in syringes. Improvements in the ease and use of such apparatus and equipment would be desirable.

### BRIEF DESCRIPTION OF THE DRAWINGS

The drawings described herein are for illustration purposes only and are not intended to limit the scope of the present disclosure in any way. In the drawings, closely related figures have the same number but different alphabetic suffixes.

FIG. 1 shows an embodiment of the apparatus of the invention, as the apparatus might appear to medical personnel.

FIG. 1a shows the apparatus of FIG. 1 with a syringe or container having first and second ends placed on the transparent tray.

FIG. 1b shows an exploded schematic head-on view of the apparatus of FIG. 1 looking from the front of the apparatus showing one embodiment of elements for analyzing a vial's label including analyzing barcodes on the label.

FIG. 1c shows a schematic of a direct head-on view similar to FIG. 1b from the front of the apparatus showing one embodiment of elements used for analyzing a syringe or container having first and second ends.

FIG. 1d shows a side view schematic of the apparatus of FIG. 1 with the viewer looking on from the right side of apparatus. The schematic shows the side view of camera (44) looking from the right side, the syringe (30), the transparent tray (25), the color number one transparency (one of two) (36) or filter looking on from the right side of apparatus (21), and the bottom light source (38) of the light assembly of the apparatus (21) of the invention of FIG. 1.

FIG. 2a shows one embodiment of the view of the camera of the apparatus of FIG. 1 looking downwards onto the syringe or container having first and second ends full of clear liquid.

FIG. 2b shows one embodiment of the view of the camera of the apparatus of FIG. 1 looking downwards onto the syringe or container having first and second ends full of clear liquid as the syringe or container having first and second ends is placed off center to the left.

FIG. 2c shows an embodiment of the camera's view of the apparatus of FIG. 1 looking downwards onto the syringe or container having first and second ends full of clear liquid with an air bubble present. The air bubble (51) is straddled between first and second colors of light projecting through the syringe or container having first and second ends.

FIG. 2d shows an embodiment of the camera's view of the apparatus of FIG. 1 looking downwards onto a syringe (30), or container having first and second ends, full of clear liquid with two air bubbles (52) and (53) present. Air bubble (52) is surrounded by the color one of two or the first color. Air bubble (53) is surrounded by the color two of two or the second color.

FIG. 2e shows an embodiment of the camera's view of the apparatus of FIG. 1 looking downwards onto the syringe, or container having first and second ends, full of air.

FIG. 3a shows an embodiment of a light ray diagram showing how two colors of light travel upwards through a syringe full of clear liquid and how the first and second colors of the light are transposed before being viewed by a camera of the apparatus of FIG. 1.

FIG. 3b shows a close-up view of the light refractions of the clear liquid filled syringe area of the light ray diagram of FIG. 3a.

FIG. 3c shows an embodiment of a light ray diagram of how two colors of light travel upwards through a syringe full of air and how the light colors are not transposed before being viewed by a camera of the apparatus of FIG. 1.

FIG. 3d shows a close-up view of the air filled syringe area of the light ray diagram of FIG. 3c.

FIG. 3e shows an embodiment of a light ray diagram of how two colors of light travel upwards through a bubble of air in a liquid filled cylinder and how the light colors are not transposed before being viewed by the camera of the apparatus of FIG. 1.

FIG. 3f shows a close-up view of the bubble of air area of the light ray diagram of FIG. 3e.

FIG. 4a shows an embodiment of the camera's view of the apparatus of FIG. 1 looking downwards onto the medical dosage device full of Cloudy (White) opaque insulin properly mixed. The colors of light are not able to penetrate the Cloudy (White) opaque insulin properly mixed (66) before being viewed by the camera (34) of the apparatus of FIG. 1a. The length of Cloudy (opaque) insulin in cylinder properly mixed (67) and width of Cloudy (opaque) insulin in cylinder properly mixed (68) are shown, and thus the volume of Cloudy (White) opaque insulin properly mixed (66) can be scanned and measured.

FIG. 4b shows an embodiment of the camera's view of the apparatus of FIG. 1 looking downwards onto the medical dosage device partially filled with Cloudy (White) opaque insulin properly mixed (66) and partially filled with Cloudy insulin whose suspension has settled—becoming clear instead of opaque and letting light thru (69). The length of liquid in cylinder (47) and the width of liquid in cylinder (49) can be measured and thus the volume of liquid in the syringe or container having first and second ends can be evaluated by the apparatus of FIG. 1.

FIG. 5 shows an embodiment of a flowchart delineating the operation of the software of the apparatus FIG. 1.

FIG. 6A(1) shows an embodiment of a flow diagram delineating the software flow of the login procedure, medicine type verification, administrative tasks and connectivity of the EHR of the patient of the apparatus of FIG. 1.

FIG. 6A(2) shows an embodiment of a flow diagram delineating the software flow of the patient selection, placing syringe on device and data logging of the apparatus of FIG. 1.

FIG. 6B shows an embodiment of a flow diagram delineating the software flow of verifying that the proper dose of medicine is in the syringe, and considering air bubbles, of the apparatus of FIG. 1.

FIG. 6C shows an embodiment of a flow diagram delineating the software flow of printing a label if the dose in the syringe is correct of the apparatus of FIG. 1.

FIG. 7 shows an embodiment of the apparatus of FIG. 1 receiving real time data from the Electronic Health Record (230) of a patient.

FIG. 8 shows an embodiment of a culling process of the data received from the Electronic Health Record (230) of a patient.

FIG. 9 shows an embodiment of a computer processor (24) of the apparatus of FIG. 1, parsing narrative data (241) into correct doses for different conditions (242) from a medication order (240) of the patient.

FIG. 10 shows an embodiment of a computer processor (24) of the apparatus of FIG. 1 determining that only one condition exists (252) by reading from a laboratory order (250) of the patient.

FIG. 11 shows an embodiment of a computer processor (24) of the apparatus of FIG. 1 determining that multiple conditions exists (261,262) by reading from a laboratory order (250) of the patient.

FIG. 12 shows an embodiment of a concept flow diagram delineating the processing operation of the data from the portal feed of the Electronic Health Record (230) of a patient by the apparatus of the invention.

FIG. 13 shows an embodiment of a concept flow diagram delineating the continued processing of the saved data from initial processing (FIG. 12, #294) by the apparatus of the invention.

### DETAILED DESCRIPTION

Some embodiments of the invention can lessen medical adverse events in the preparation process for injecting medicines into patients in the hospital. Some of the ways in which said medical adverse events can occur are from an incorrect medicine being injected; or from an incorrect dosage of medicine being injected; or from air being present in an injected dose. In some current standard hospital procedures for medical injections, one medical practitioner prepares the dose in a syringe while a second medical practitioner is required for the purpose of checking said preparation process. In some embodiments, the apparatus of the invention can replace the need for said second medical practitioner by supplying the expertise to check said preparation process. The apparatus of the invention can also add the value of recording every step of each iteration of said preparation.

In the case of insulin, a diabetic patient can need at least three and maybe four injections of insulin per day while in the hospital. Effective dosage of said injections may be dependent on the most current known blood glucose level, and optionally other medical conditions or history, of the patient. In some embodiment, the apparatus of the invention can automatically retrieve the most recent known blood glucose level, and optionally other medical conditions or history, of the patient from said patient's EHR. Effective after release treatment of said patient may require the knowledge of past dosages while the patient is in the hospital be verified as correctly prescribed and verified as correctly administered, and the effect said past correctly verified dosages had on the blood glucose level, and optionally other medical conditions or history, of the patient. The apparatus of the invention can optionally have access to that data in the EHR. The invention has applicability to other medicaments, and can optionally access medical conditions of the patient, which can include the patient's medical history, for example by accessing the EHR of the patient, to provide the proper dose of a medicament to the patient.

In some embodiments, a method and apparatus are provided for determining the volume of a liquid, for example a dosage of a liquid, in a container in any suitable manner. The container can optionally be one of a plurality of known containers. The invention can optionally determine the specific container from the plurality of known containers, for example digitally stored in the apparatus of the invention or storage accessibly by the apparatus of the invention. The apparatus can optionally include at least one sensor for determining the volume of a liquid in the container. The apparatus can optionally include a sensor, such as a digital camera, for viewing the container. A processor can be electrically coupled to the camera and configured to optically detect certain characteristics of the container viewed by the camera and to access a computer memory having stored characteristics of the plurality of known containers. The processor can be configured to compare the detected certain characteristics with the stored characteristics to identify the container from the plurality of known containers. The processor can be configured to calculate the volume of the container, and optionally a volume of liquid in the container, as a function of the distance between the first and second ends of the container as viewed by the camera.

In some embodiments, a method and apparatus are provided for determining the presence of any air pockets or air, for example air bubbles, in a volume of a liquid in a container, for example a volume of liquid dosage in a container. The apparatus optionally includes a digital camera for viewing the liquid within the container and providing at least one image of the liquid within the container. A processor is electrically coupled to the camera for receiving the at least one image from the camera. The processor can be configured to determine whether the liquid contains any air pockets or air based on the at least one image. Such determination of any air pockets can optionally be utilized by the apparatus to more precisely determine the actual volume of liquid in the container, for example by subtracting the volume of the air pockets or air from the volume of the liquid in the container, for example determined as a function of the distance between the first and second ends of the container.

In some embodiments, a method and apparatus are provided to confirm the dosage of a medicament in a container in any suitable manner. The method and apparatus can optionally utilize a camera and a processor electrically coupled to the camera. The proper or prescribed volume of the dosage can optionally be accessed from a computer memory. The container can be viewed with the camera to obtain at least one image of the container. The at least one image of the container can be delivered to the processor. The processor can calculate the volume of the liquid in the container utilizing the at least one image in any suitable manner and compare the calculated volume to the proper volume.

The method and apparatus of the invention can optionally use computer vision to determine the presence and volume of liquid and air in the container by watching the refraction behavior of light through liquid and air. The method and apparatus of the invention can possibly be applied to help lessen adverse events in medicine, manufacturing, and science.

The present invention can allow an inexpensive syringe or other container to become a “smart insulin syringe or container”. The smart insulin container can know the dose and type of insulin, or other medicament, it contains as well as the patient the medicament is intended for, and can communicate this information to the Electronic Health Record (EHR) of the patient. Said smart medicament container can know what is inside itself and which patient it is intended for, and can tell the EHR that information in any suitable manner, for example through use of a simple barcode scan.

The embodiments of the invention set forth below are examples of the invention, and may in some instances be broader than the foregoing embodiments of the invention but are not intended to limit the breadth of the foregoing embodiments. Additional features of the invention set forth in such embodiments are optional. A feature of any embodiment set forth below can be combined with any of the foregoing embodiments, with or without any other feature of any embodiment set forth below. All characteristics, steps, parameters and features of the apparatus and methods below are not limited to the specific embodiments or specific parts set forth below, but instead are equally applicable to any or all of the foregoing embodiments of the invention and to all embodiments of the invention. Broad terms and descriptors are replaced herein with more specific terms and descriptors not to limit a disclosure to a specific term or descriptor but merely for ease of discussion and understanding.

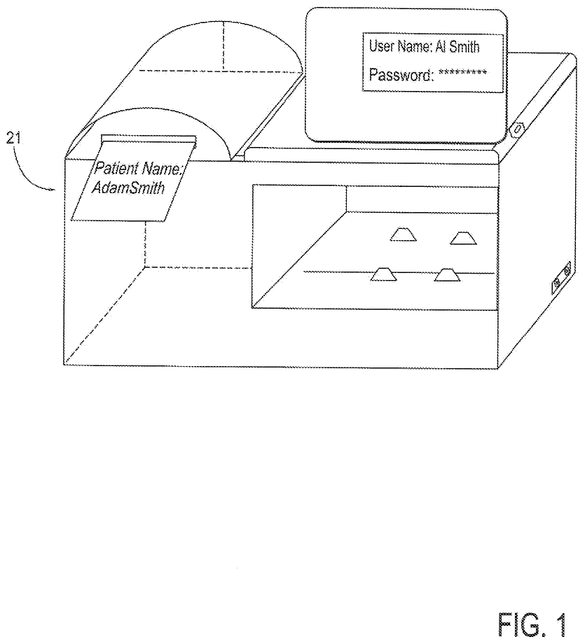

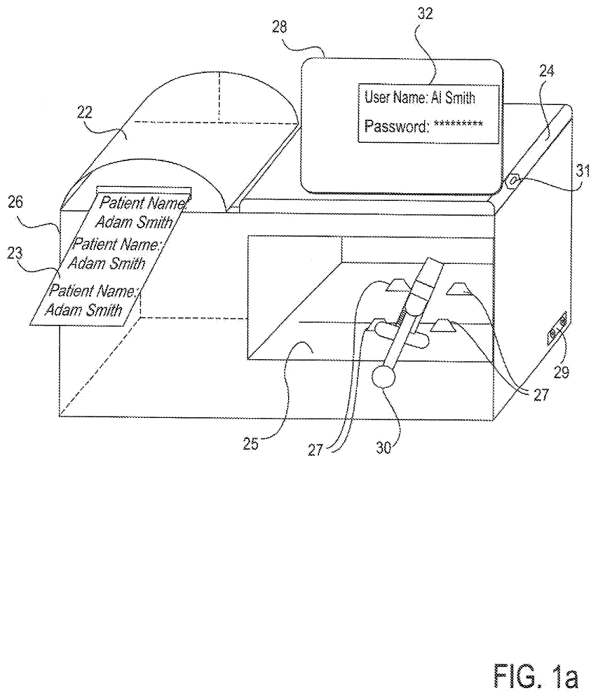

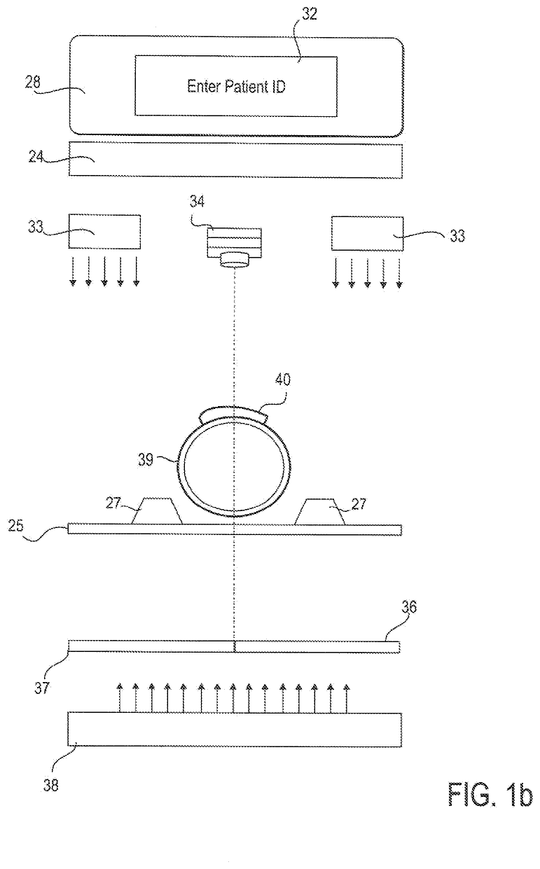

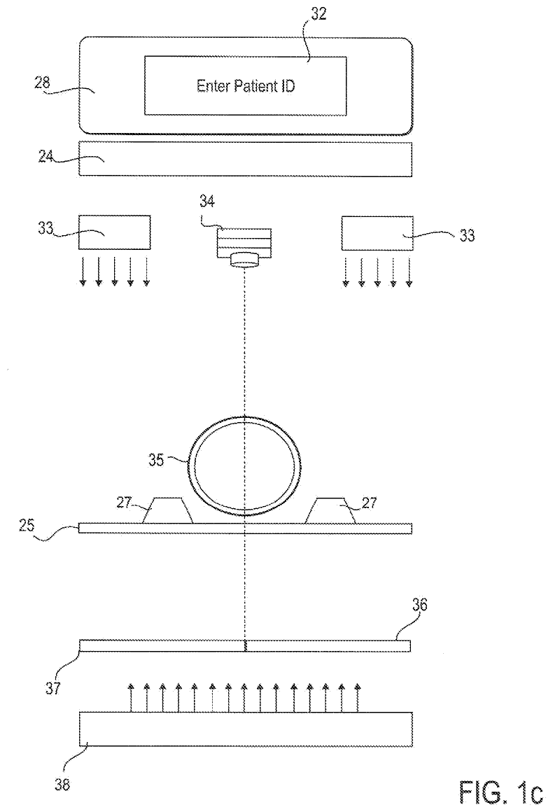

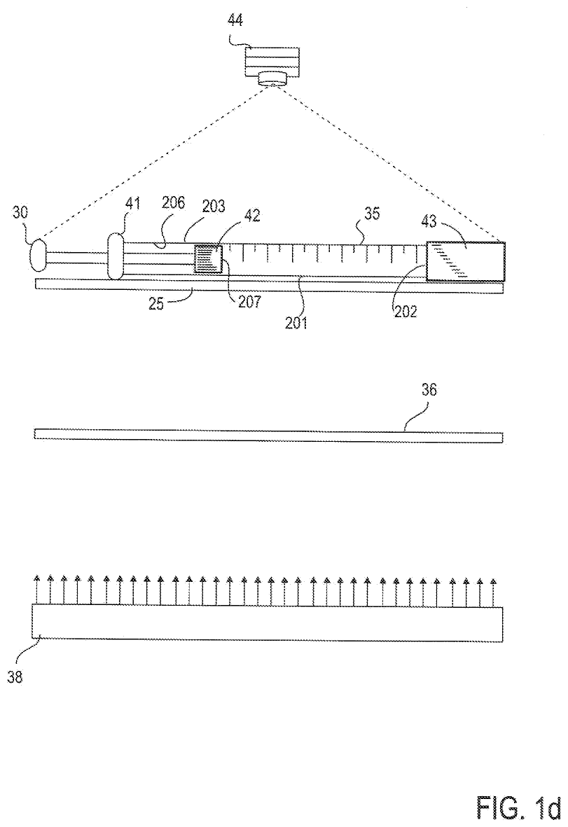

The apparatus and method of the invention can be used with any suitable container for holding a liquid. One suitable container is syringe (30). One suitable syringe includes a barrel (201) or cylinder for containing a liquid and having a first end (202) provided with a fluid exit port (not shown) and a second end (203) provided with an opening (206) (see FIG. 1d). The syringe can additionally include a suitable plunger, such as plunger (42) having an end (207) for extending through the opening (206) in the barrel (201) for slidable disposition in the barrel.

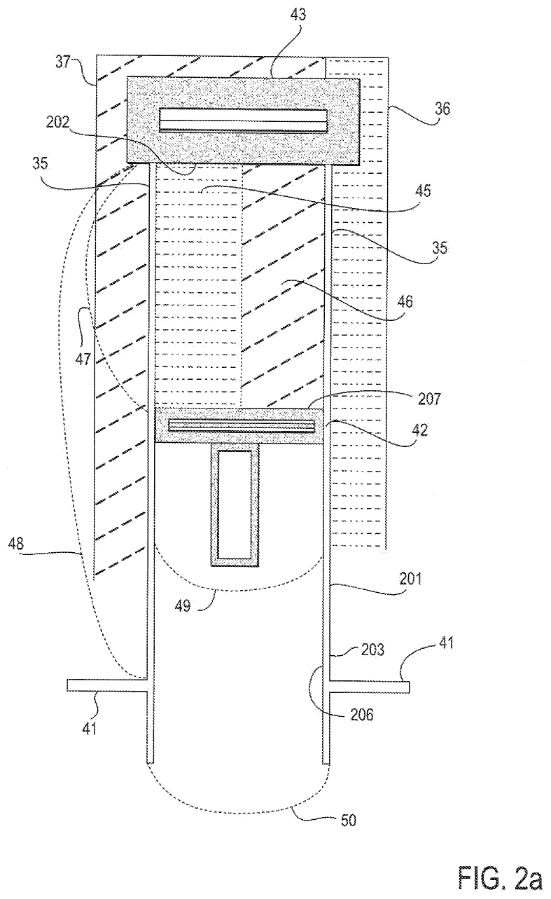

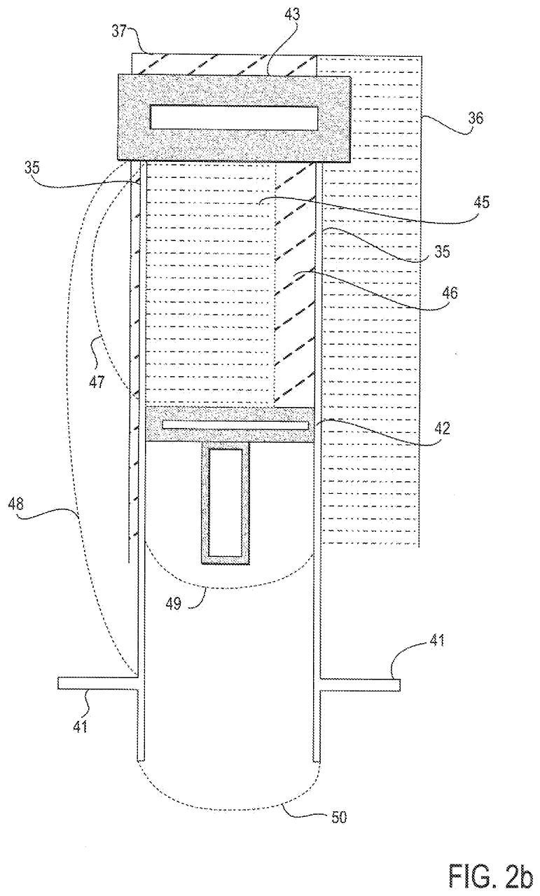

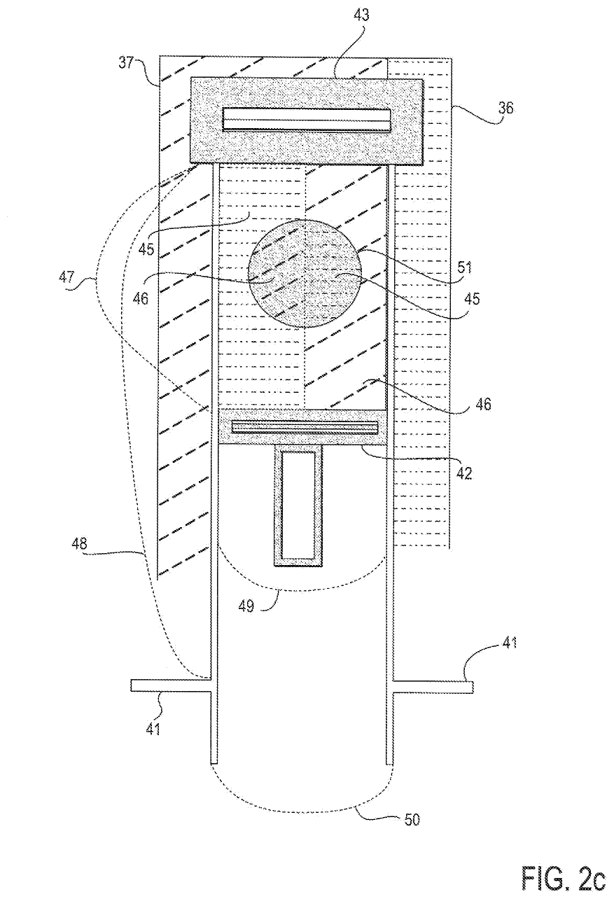

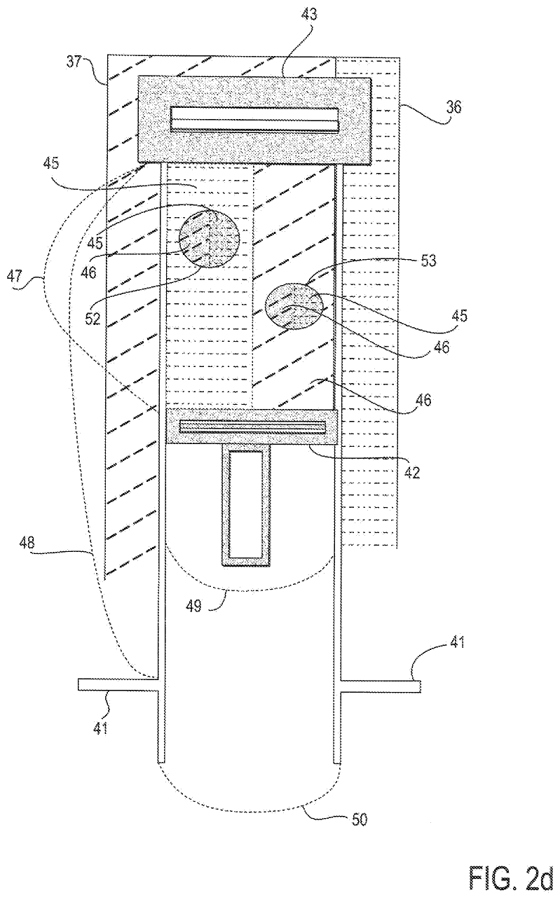

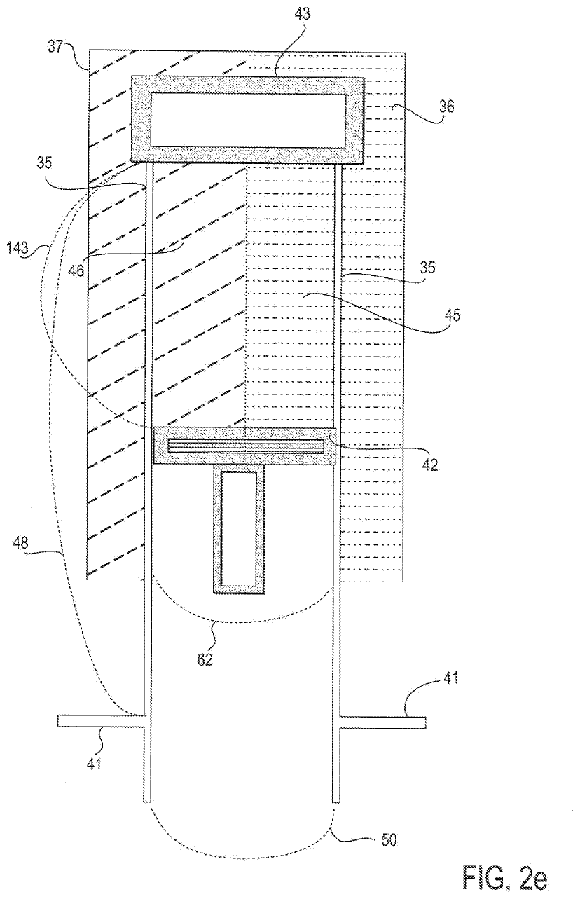

The apparatus of the invention can be of any suitable type and is not limited to the disclosure and drawings herein. An embodiment of the apparatus of the invention is illustrated in FIGS. 1 and 1a. Apparatus 21 can optionally be located in the medication room of a hospital. In FIG. 1a, labeled parts on the exterior of apparatus (21) include touchscreen (28), which can serve as a display and as an input device, graphical user interface software or GUI (32) displayed on the touchscreen (28) and a computer (24) or other suitable processor of any suitable type. In some embodiments, the computer includes a central processing unit (CPU) and memory coupled to the CPU. The memory can include any suitable storage memory, for example hard drives and/or solid state drives, as well as short term member such as random access memory. The computer can be programed in any suitable memory, including by means of software or firmware. Any such software can be stored in the memory of the computer 24, or in the memory of a remote computing device in communication with the computer 24. FIG. 1a also labels the exterior housing (26) or structure of the apparatus (21), the printer (22) which can printout the attachable label (23) with all pertinent data and scan codes. Said label (23) can be attached by medical personnel to the syringe (30) or container having first and second ends. FIG. 1a labels the power supply (29), and also the data connection port (31) to be used for receiving and transmitting data. FIG. 1a labels the transparent tray (25) on which the medicine vial or syringe (30) or other suitable container having first and second (202,203) ends can be placed in order for the computer (24) to process said medicine vial or syringe (30) or container having first and second ends. FIG. 1a also labels the guides (27) on top of the tray (25). Said guides can be used so the medical practitioner visually knows where the syringe (30) or container having first and second ends is to be placed horizontally. In some embodiments, the syringe (30) or container having first and second ends on the tray (25) such that cap (43) or first end (202) is inserted first into the apparatus (21) and onto the tray 25. In some embodiments, the syringe (30) or container having first and second ends is placed on the tray 25 so that both the cap (43) or first end and the wings or flanges (FIG. 2a, #41) of the syringe (30) or container having first and second ends are contained by the front and back boundaries of the tray (25).

Standard hospital insulin preparation process, which is to be checked and recorded by the apparatus (21), starts with examining whether the correct medicine is being used.

FIG. 1b shows an embodiment of a frontal head-on two dimensional cross section schematic top-to-bottom view of the positioning of the elements of the apparatus (21) that may come into play in order to analyze whether the correct medicine is present. In some embodiments, the analysis of apparatus (21) is commenced by having the medical practitioner place the vial or container of medicine (39) on the transparent tray (25) between the guides (27) under the camera (34) with the barcode facing upward towards the camera. Standard barcode reading techniques can then be used by the computer (24) while receiving the image from the camera (34). The apparatus (21) can alert medical personnel quickly and easily, for example thru GUI (32), if an incorrect medicine is present before the medicine is administered to the patient.

FIG. 1b also shows a frontal head-on two dimensional cross section schematic top-to-bottom view of an embodiment of the positioning of the elements of the apparatus (21) that may come into play in order to analyze whether the medicine is current or expired. In some embodiments, the analysis of apparatus (21) is commenced by having the medical practitioner place the vial of medicine (39) on the transparent tray (25) between the guides (27) under the camera (34) with the barcode facing upward towards the camera. Standard barcode reading techniques can then be used by the computer (24) while receiving the image from the camera (34). If the barcode does not yield the expiration date information, the computer (24) can use standard character recognition of printing on the label (40) to decipher the expiration date. If standard character recognition does not yield satisfactory results, the medical practitioner can be asked, for example by the GUI (32), to enter the expiration date that is on the label using the touchscreen (28) or any other suitable input technique and device. The apparatus (21) can thus alert medical personnel quickly and easily if an expired medicine is present before being administered to the patient.

FIG. 1c shows a frontal head-on two dimensional cross section schematic top-to-bottom view of an embodiment of the positioning of the elements of the apparatus (21) that can come into play in order to analyze whether the cap or first end is on the syringe (30) or container having first and second ends, and whether the correct syringe (30) or container having first and second ends is present, and whether the dosage in the syringe (30) or container having first and second ends is correct. In some embodiments, the analysis of apparatus (21) is commenced by having the medical practitioner place the syringe (30) or container having first and second ends on the transparent tray (25) between the guides (27). In some embodiments, the syringe (30) or container having first and second ends can be placed on the tray (25) such that cap (FIG. 1d, #43) or first end is inserted first into the apparatus (21) and onto the tray 25. In some embodiments, the syringe (30) or container having first and second ends is placed on the tray 25 so that both the cap (FIG. 1d, #43) or first end and the wings or flanges (FIG. 2a, #41) of the syringe (30) are contained by the front and back boundaries of the tray (25). Such placement allows the camera to see said wings or flanges (FIG. 2a, #41) and said cap (FIG. 1d, #43) or first end to later decipher the size of the syringe or container having first and second ends.

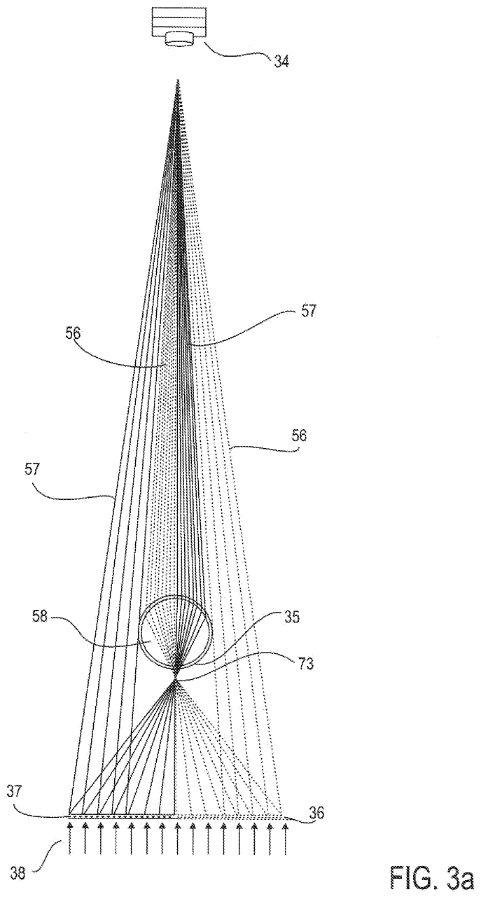

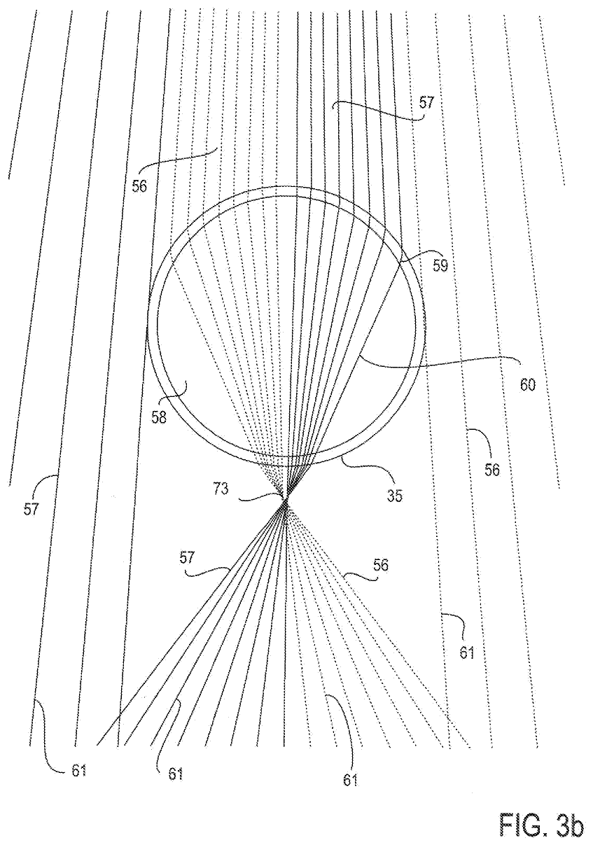

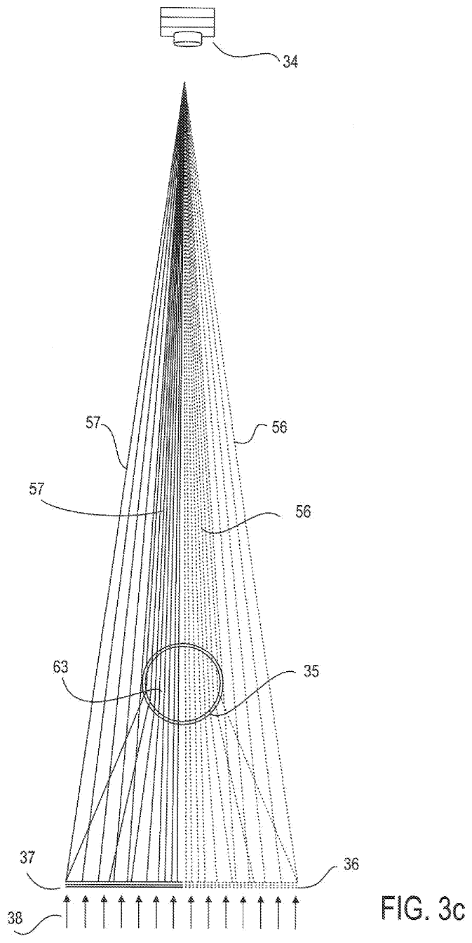

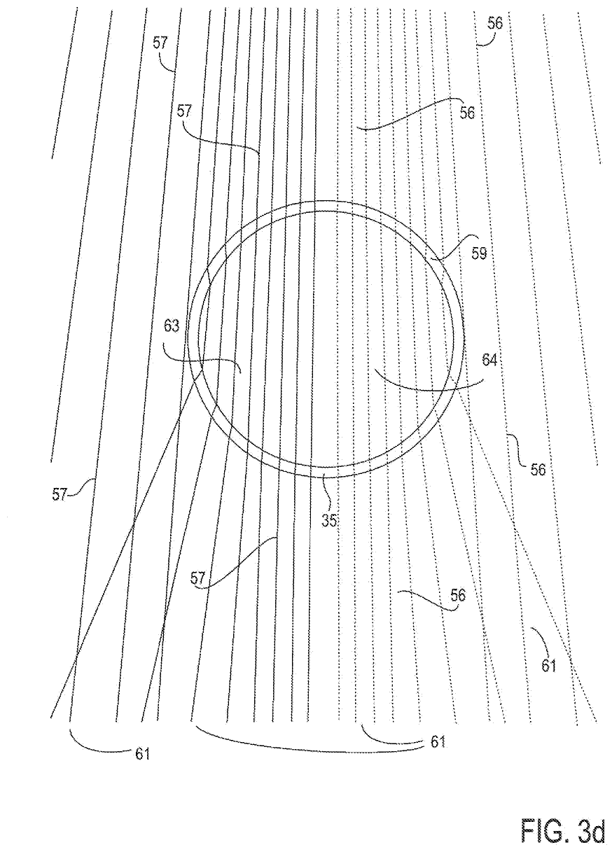

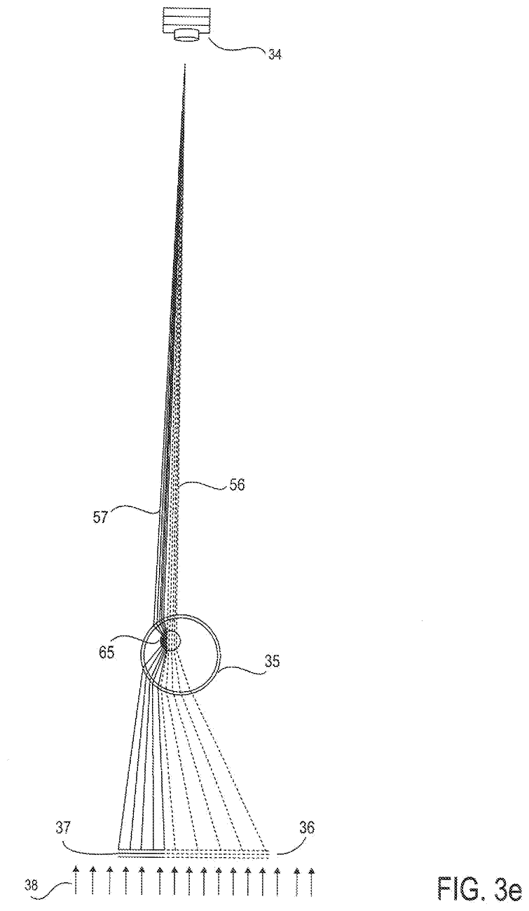

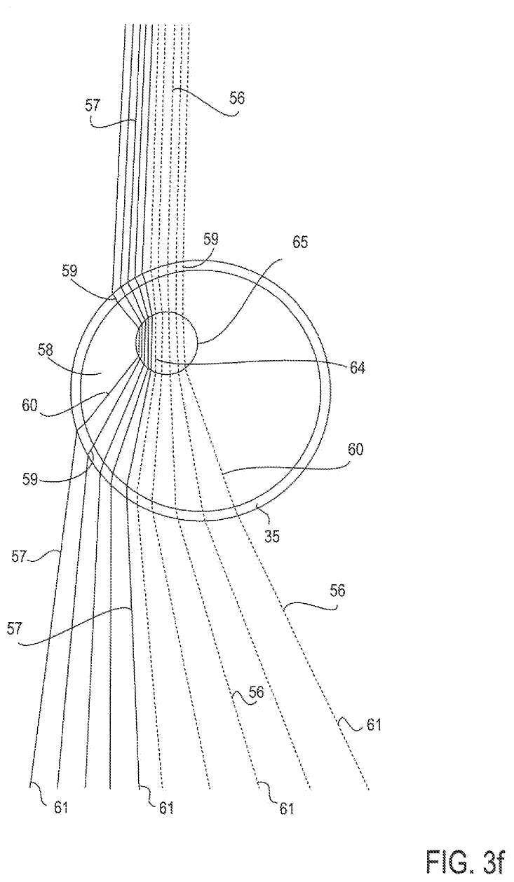

In some embodiments, the camera (34) can take a picture using top lighting (33). Standard computer vision software in the computer (24) can use object identification to decide whether the cap (FIG. 1d, #43), or first end, is present or is not present on top of the syringe (30) or container having first and second ends. In some embodiments, the cap or first end is required to be on. Analysis procedures of the apparatus (21) concerning whether the correct syringe (30) or container having first and second ends (202,203) is present, and whether the dosage in the syringe (30) or container having first and second ends is correct, is discussed later in this text with respect to the FIG. 2 series and the FIG. 3 series of drawings. For now, please note that in an embodiment the line where the Color Number One Transparency (36) or filter meets the Color Number Two Transparency (37) or filter is ideally aligned with the center of the syringe (30) or container having first and second ends and the center of the lens of the camera. This is one preferred placement situation, although it is appreciated that the syringe or container having first and second ends may be placed off center in the operation of the apparatus (21), as will be discussed later when referring to FIGS. 2b and 2c.

FIG. 1d shows an embodiment of a side view, two dimensional cross section schematic of the positioning of elements of the apparatus (21) and the syringe (30) or container having first and second ends. FIG. 1d is looking directly towards the right side of the apparatus (21) so that the rear of the apparatus (21) is to the right of the image and the front of the apparatus (21) is to the left of said image. FIG. 1d shows that the Color Number One Transparency (36) or filter, and by implication also the Color Number Two Transparency (37) or filter which is blocked from view, are in some embodiments at least as long as the longest syringe (30) or container having first and second ends being testing on the apparatus (21). In some embodiments, the depth of the tray (25) can be at least as long as the longest syringe (30) or container having first and second ends testing on the apparatus (21). Compare FIG. 1d with both FIGS. 1a and 1c to get two perspectives on the positioning of syringe (30) or container having first and second ends and the positioning as well as size of Color Number One Transparency or filter (36) and Color Number Two Transparency (37) or filter and tray (25).

FIG. 2a portrays an embodiment of the view the camera (34) of the following: a clear liquid filled syringe (30) or container having first and second ends, the Color Number One Transparency (36) or filter, and Color Number Two Transparency (37) or filter. The bottom lighting (38) is in effect but the camera does not see the actual bottom lighting apparatus (38) which is below the transparencies or filters.

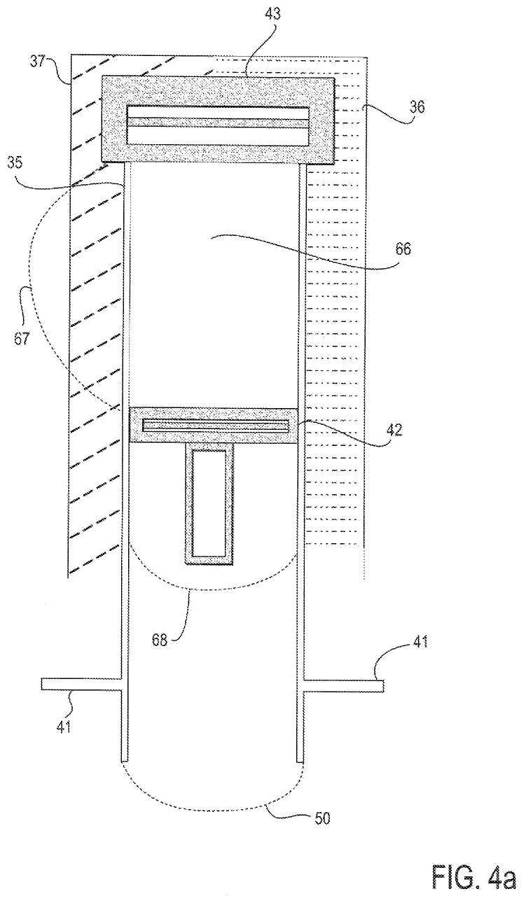

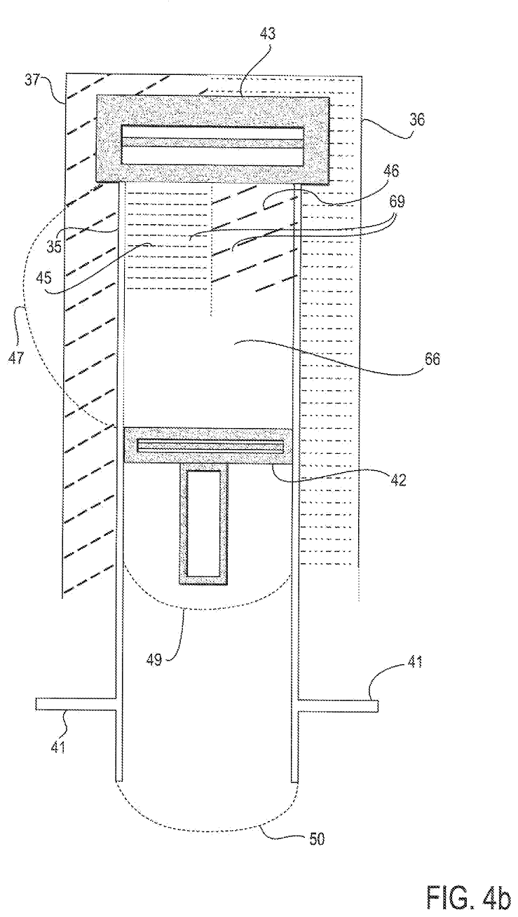

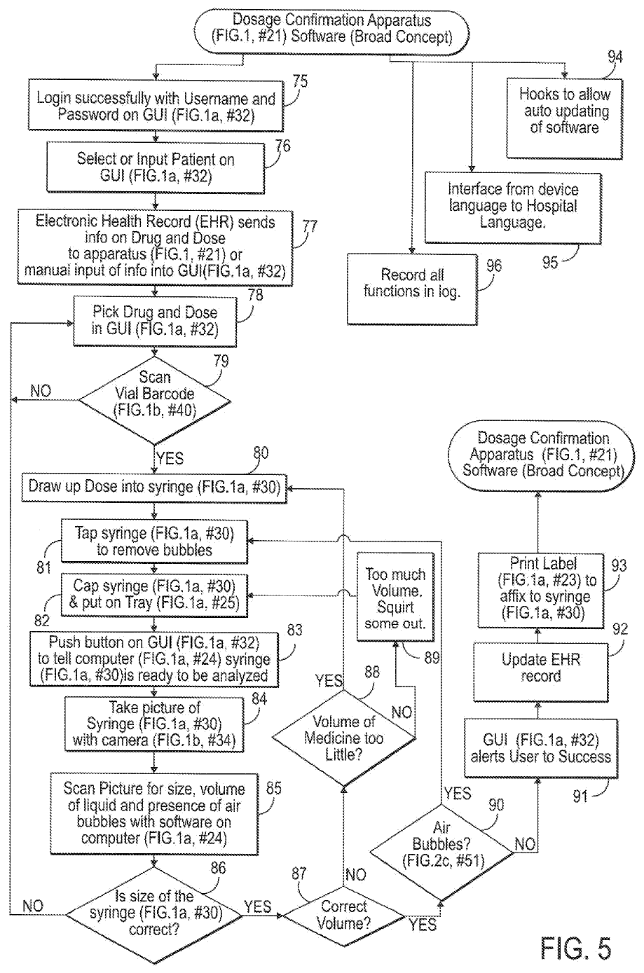

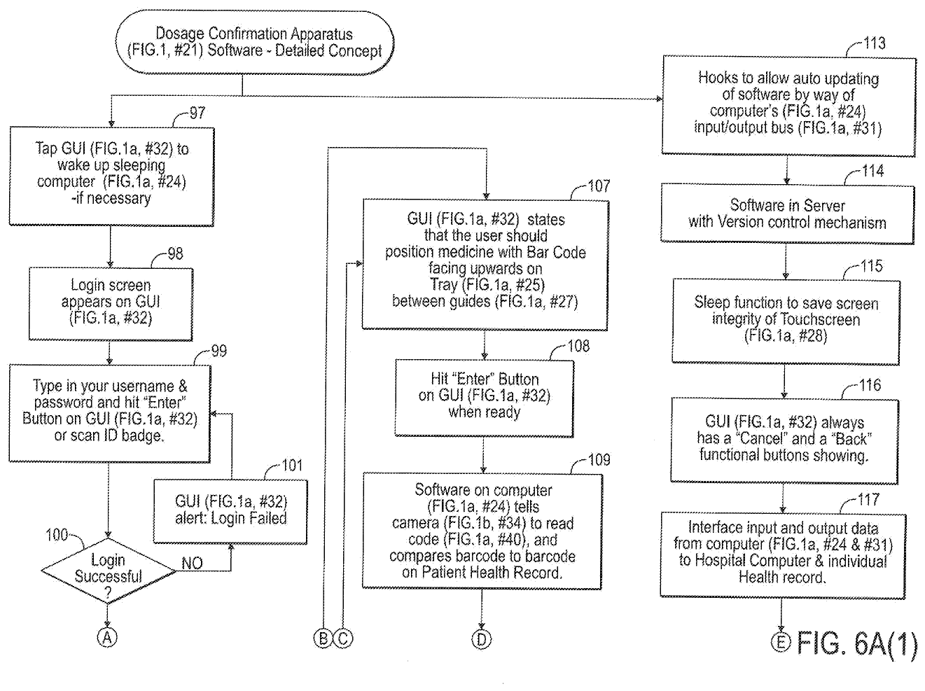

In some embodiments, the apparatus (21) can determine whether the syringe (30), or container having first and second ends, is the correct size of syringe, or container having first and second ends, for the desired dose. In some embodiments, the process of the computer (24) which decides on the correctness of the size of the syringe, or container having first and second ends, determines the length of the cylinder (48). The length of the cylinder (48) can be defined as the distance from where the bottom of the cap (43) or first end meets the wall of syringe cylinder (35) to where the wings or flanges (41) meet the cylinder wall (35). Standard computer vision programming of computer (24) can use object recognition to determine exactly where said two points are, and then computer (24) can calculate the length of the cylinder (48). Once the length of the cylinder (48) has been calculated, computer (24) can compare said calculated length with known cylinder lengths of different size syringes, or containers having first and second ends, and decide if the desired syringe, or container having first and second ends, is in place and report to medical personnel through GUI (32) or otherwise. In some embodiments, the computer can store in memory the length of the cylinder of a finite number of syringes, or containers having first and second ends. Most of the said syringe cylinder length data in memory has the property of one unique length per type of syringe, in which case syringe type can be ascertained by calculating the length of the syringe cylinder and associating that length in the stored library of data with a stored type of syringe. It is appreciated that differing syringe or container having first and second ends types may have the same cylinder length and such types should be noted in the computer's stored data and should contain additional differentiating data such as cylinder width (50) (see FIG. 4b). Such length, or other characteristics of the cylinder or other parts of a finite number of syringes, can be stored in any suitable location, such as in memory in any suitable location. Such memory, or storage, can be included in the apparatus or located remote or offsite from the apparatus.

In some embodiments, the apparatus (21) determines whether there is only liquid in the syringe (30) or container having first and second ends, or conversely, whether there is air in the syringe or container having first and second ends. FIG. 2a portrays a syringe (30), or container having first and second ends, filled with liquid. Computer (24) can notice the transposing positions of color when liquid is in the cylinder. In some embodiments, the color one of two (45), or first color, is on the left of the cylinder while the transparency creating that color (36) is on the right of the cylinder. Accordingly, the color two of two (46), or second color, is on the right of the cylinder while the transparency or filter creating that color (37) is on the left of the cylinder.

In some embodiments, two distinct colors for the transparencies (36,37) are utilized, such that the color hue value of each color can be used in addition to saturation and intensity values to distinguish the two colors. Use of color hue values can be an improvement over using strictly light intensity in the computer vision analysis algorithms, as for example would be the case if black and white were used. Use of color hue values can improve the ability of the algorithm to distinguish between the two colors in varying ambient light conditions, both improving accuracy and repeatability of the measurements.

The physics of transposition of colors through a liquid are explained below in the discussion of the FIG. 3 series of illustrations. The apparatus (21) can tell that the cylinder in FIG. 2a is filled with liquid by computer (24) discerning the existence of the transposed colors which appear in an orderly transposition throughout the cylinder.

Once the apparatus (21) has decided that there exists a cylinder full of liquid, the apparatus (21) can measure the volume of liquid inside the cylinder in order to decide if the correct dosage is present. Looking at what FIG. 2a portrays, computer (24) can discern the length of the liquid (47), defined as the distance from where the bottom of the cap (43) or by the first end (202) of the barrel (201) extending between the wall of syringe cylinder (35), to where the top of the plunger (42) or second end (207) extends between the cylinder wall (35). Standard computer vision software in computer (24) can use object recognition to determine exactly where said two points are, and then computer (24) can calculate the length of the liquid (47) between such two ends (202) and (207). To measure the width of the liquid, computer (24) can be programmed with the width of liquid in known syringe sizes, or known container having first and second ends sizes. Alternatively, computer (24) can count the pixels of color from the inside edge of the left cylinder wall (35) to the inside edge of the right cylinder wall (35). Computer (24) can translate the number of pixels into distance and thereby discerning the width of the liquid (49). Once the length and width of the liquid is known, volume can be calculated by the formula

L*Πr²

where L=length of liquid in cylinder (48) and r=0.5*width of liquid in cylinder (49).

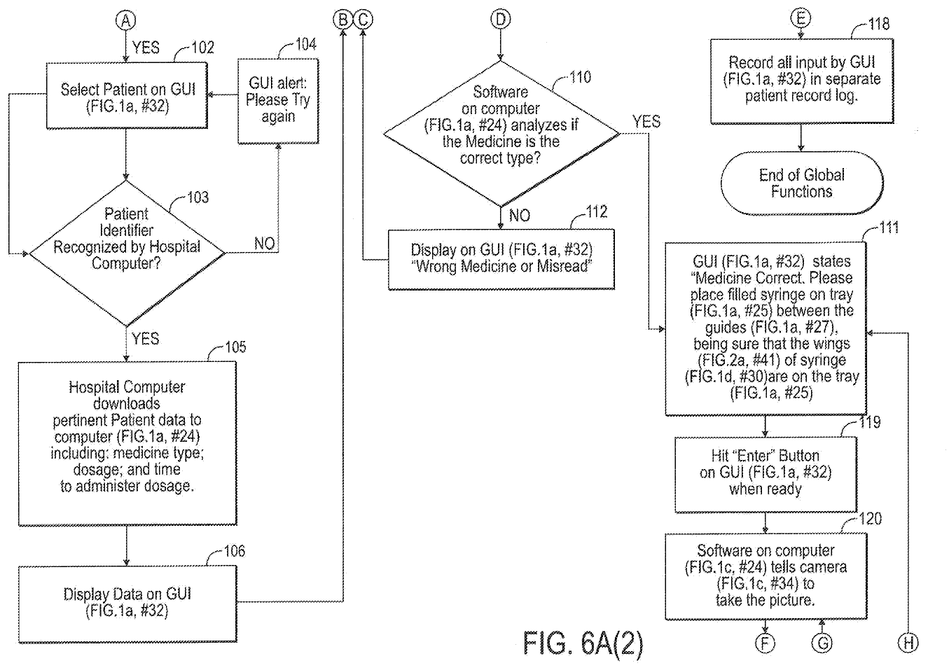

The computer (24) can then check to see if said volume coincides with the desired volume of medicine. If the volume coincides with the desired volume of medicine, the medical personnel is alerted through GUI (32) or otherwise and the process is allowed to continue. Apparatus (21) can alert medical personnel thru GUI (32) or otherwise if an incorrect dosage of medicine is present and suggestions can be made through GUI (32) or otherwise to correct the situation (see for example the bottom of FIG. 5 for “Correct Volume” decision.)

In some embodiments, the apparatus (21) can allow for a certain amount of horizontal leeway when placing the syringe (30), or container having first and second ends, on the top of tray (25) in between the guides (27). FIG. 2b shows an embodiment of the view of the camera (34) looking downwards onto the syringe (30) or container having first and second ends full of clear liquid as the syringe (30) or container having first and second ends is placed to the left, off center of the line between the two transparencies or filters. Color one of two (45) or first color is on the left of the cylinder while the transparency or filter creating that color (36) is on the right of the cylinder. FIG. 2b shows the transposing positions of color when liquid is in the cylinder, but with differing amounts of each color. Accordingly, the color two of two (46) is on the right of the cylinder while the transparency creating that color (37) is on the left of the cylinder. The process of transposition of colors through a liquid will be explained below in the discussion of the FIG. 3 series of illustrations. The apparatus (21) can determine that the cylinder in FIG. 2b is filled with liquid by computer (24) discerning the existence of the transposed colors which appear in an orderly transposition throughout the cylinder, even if the syringe or container having first and second ends is off center. In some embodiments, the existence of both of the two transposed colors prove liquid is in the cylinder, no matter what the ratio of color one (45) or first color is to color two (46) or second color. Thus there is a certain amount of play in the left or right positioning of the syringe or container having first and second ends, which makes it easier for the medical personnel to use the apparatus (21). The ratio of colors produced by a syringe (30) or container having first and second ends placed off the center line of the two transparencies to the right instead of the left would similarly still be able to be analyzed by the apparatus (21). The play in the left and right positioning of the syringe (30) or container having first and second ends is increased as the syringe (30) or container having first and second ends is moved a greater distance above the focal point (73) seen in FIG. 3a and FIG. 3b.

In some embodiments, the apparatus (21) detects if air is in the syringe (30) or container having first and second ends. In the case of insulin, the air itself may not be dangerous to the patient, as insulin is not an intravenous injection. However, in said case of insulin, the air may be of significant volume to affect the correct measurement of the volume of medicine in the syringe (30), or container having first and second ends, by displacing said medicine enough to affect measurement of the true amount of medicine. The apparatus (21) can alert medical personnel thru GUI (32) or otherwise if a large enough amount of air is present in the medicine that would affect the volume measurement of said medicine prior to being administered.

In the case of intravenous injections, air within the syringe (30), or container having first and second ends, can be dangerous to the patient. The apparatus (21) can alert medical personnel if any potentially harmful amount of air is present in injectable medicine prior to being administered intravenously. FIG. 2c shows an embodiment of the camera's view looking downwards onto the syringe (30) or container having first and second ends full of clear liquid with an air bubble (51) present. The air bubble (51) is straddled between two colors. The colors of light are transposed in the liquid before being viewed by the camera of the apparatus of the invention. Where the air bubble exists, in some embodiments, the colors of light are not transposed before being viewed by the camera of the apparatus of the invention. Thus said air bubble can be discerned, measured, and reported to medical personnel. See discussion below with respect to FIG. 3e and FIG. 3f for the physics of this colored lighting phenomenon. If two or more air bubbles are totally aligned or one air bubble obscures another air bubble which is below the top air bubble, in an embodiment the software can use enough leeway in calculations of area in order to decide on the potential of an unwanted amount of air. If two or more air bubbles are partially aligned, in some embodiments the software can use extrapolation from the visible circumferences in order to measure the area of air and decide on the potential of an unwanted amount of air.

Unlike an air bubble (51) straddled between two colors, air bubbles can also appear enclosed within one color. FIG. 2d shows an embodiment of the camera's (24) view looking downwards onto the syringe (30) or container having first and second ends full of clear liquid with two air bubbles (52,53) present. Air bubble (52) is surrounded by the color one of two or first color. Air bubble (53) is surrounded by the color two of two or second color. The colors of light are transposed in the liquid before being viewed by the camera of the apparatus of the invention, however where the air bubbles exist, the colors of light are not transposed before being viewed by the camera. Thus the bubbles can be discerned, measured, and reported to medical personnel.

In some embodiments, the apparatus (21) determines if the syringe or container having first and second ends is full or partially full of air. FIG. 2e shows an embodiment of the camera's (24) view looking downwards onto the syringe (30) or container having first and second ends full of air. The colors of light are not transposed as the colors were transposed in liquid before being viewed by the camera of the apparatus of the invention. The length of the air (143) and width of the air (62) are shown, and thus the air can be discerned and measured.

The physics of light behavior in air and clear liquid and plastic are used by the apparatus (21) to distinguish the existence of clear liquid within the syringe (30) cylinder, or container having first and second ends, and to distinguish the existence of air within the syringe (30) cylinder or container having first and second ends. FIG. 3a shows an embodiment of a light ray diagram depicting how two colors of light can travel upwards through a cylinder filled with clear liquid (58) and how the light colors are transposed before being viewed by the camera (34) of apparatus (21). The light emanates as white light from the bottom light source (38) and travels upward through one of the two colored transparencies (36,37), or filters, where the white light becomes colored light rays (56,57) by the respective transparency or filter.

The light that travels upward from the bottom light source (38) is omnidirectional and as the light passes through the colored transparencies (36,37) the light remains omnidirectional. The light rays shown in the FIG. 3 drawings are a subset of the infinite amount of light rays being produced. The subset of light rays shown in the FIG. 3 drawings are intended to show the area of light rays which are within the camera's (34) area of view.

FIG. 3b shows a close-up view of the light refractions of the clear liquid filled cylinder (58) area of the light ray diagram of FIG. 3a. The viewable colored light either passes by the cylinder and is not affected by the cylinder, or the viewable light passes through the cylinder filled with liquid and is refracted (59) according to the refraction index of the plastic wall of the cylinder (35) and the refraction index of the liquid (60) in the clear liquid filled cylinder (58). The light rays (56,57) transpose as they pass through the cylinder and before they reach the camera (34).

FIGS. 3a, 3b, 3c, 3d, 3e, and 3f are all light ray diagrams of an embodiment of the invention. Said light ray diagrams are based on the following:
1. Camera (34) is 3 inches above syringe or container having first and second ends.
2. The syringe (30) cylinder, or container having first and second ends, diameter is ¼″
3. The syringe (30) cylinder, or container having first and second ends, is 1″ above transparencies (36,37).
4. Wall thickness of syringe, or container having first and second ends, is 5% of overall diameter.
5. Index of refraction for syringe wall made of PET polypropylene is 1.575
6. Index of refraction for clear liquid insulin is 1.56

FIG. 3c shows an embodiment of a light ray diagram depicting how two colors of light rays (56,57) travel upwards through a cylinder filled with air (63) and how the light's colors are not transposed before being viewed by the camera (34) of the apparatus (21) of the invention of FIG. 1. The light emanates as white light from the bottom light source (38) and travels upward through one of the two colored transparencies (36,37) or filters where the white light becomes colored light rays (56,57) by the respective transparency or filter.

FIG. 3d shows a close-up view of the light refractions of the air filled cylinder (63) area of the light ray diagram of FIG. 3c. The viewable colored light either passes by the cylinder and is not affected by the cylinder, or the viewable light passes through the cylinder filled with air and is diffracted according to the refraction index of the plastic wall of the cylinder (34) and the refraction index of the air (64) in the air filled cylinder (63). The refraction index of air at standard temperature and pressure is 1.000277. The light rays (56,57) do not transpose as they pass through the air filled cylinder (63) and before they reach the camera (34).

FIG. 3e shows an embodiment of a light ray diagram depicting how two colors of light rays (56,57) travel upwards through a bubble of air (65) in a liquid filled cylinder and shows how the light rays behave if there is a bubble of air in the liquid. The light emanates as white light from the bottom light source (38) and travels upward through one of the two colored transparencies (36,37) or filters where the white light becomes colored light rays (56,57) by the respective transparency or filter.

FIG. 3f is a close up of the bubble area shown in FIG. 3e where light refractions of the air bubble within the liquid in a cylinder occur. FIG. 3f shows that the light rays (61) coming upwards from below the cylinder are refracted first by the cylinder wall (35) and then are refracted in the liquid insulin (60), and then are refracted (64) by the air in the bubble (65).

The light ray then exits the top of the bubble and is refracted again in the liquid and refracted yet again by the upper wall of the cylinder (35). The colors which pass through the bubble of air (65) are not transposed before being viewed by the camera (34) of the apparatus (21) of the invention of FIG. 1. See also FIGS. 2c and 2d for the camera's (34) view of air bubbles within liquid.

The light that travels upward from the bottom light source (38) is omnidirectional and as the light passes through the colored transparencies (36,37), or filters, the light remains omnidirectional. The light rays shown in FIGS. 3e and 3f are a subset of the infinite amount of light rays being produced. The subset of light rays shown in FIGS. 3e and 3f drawings are intended to show the area of the subset of light rays which both pass through the bubble and are within the camera's (34) area of view. Light rays which pass only through liquid and are within the camera's (34) area of view are not shown in FIGS. 3e and 3f.

On occasion, the medical personnel may be using cloudy insulin which, when mixed properly may be opaque and not permit the light rays to pass through the insulin so as to be seen by the camera (34). If allowed to settle, cloudy insulin may partially let light thru. FIG. 4a shows an embodiment of what the camera (34) may see if the syringe or container having first and second ends is filled with properly mixed cloudy insulin. Since light rays are not permeating the fluid, the camera (34) sees solid white in the cylinder. In this instance (4a), the computer (24) can easily find the length and width of the opaque medicine by using standard computer vision software (see volume measurement discussion on FIG. 2a above).

FIG. 4b shows an embodiment where the cloudy insulin has been allowed to partially settle. Where the cloudy insulin permits the partial passage of light, the transposition of light can occur as in a clear liquid (see discussion on transposition of light for FIG. 2a above.). In the situation of FIG. 4b, the apparatus (21) knows how to measure the volume of opaque liquid and clear liquid (see discussion above with respect to FIGS. 4a and 2a).

The apparatus (21) can print a label (23) marked with relevant data and containing a scan code which is to be attached by the medical personnel to syringe (30) or container having first and second ends. Creation of a label (23) can occur upon confirmation of correct dosage and correct medicine and the absence of significant air bubbles in the liquid. The data on label (23) can include, for example, the patient's name and identification, the medical personnel name and identification, the amount of the dose, the current time, the time dosage is due, the unique identification of said embodiment of apparatus (21), the patient database readable barcode with patient, drug, and dose information or any combination of the foregoing.

In some embodiments, the method of apparatus (21), from the point of view of a user, starts as the nurse or medical practitioner logs in (75) on the touchscreen (28). The nurse or medical practitioner enters the name of the applicable patient (76) on the touchscreen (28). The nurse or medical practitioner holds the medicine vial (39) underneath the camera (34) with barcode (40) face up (79). The apparatus (21) decides if the medicine is correct, and if said medicine is correct, the touchscreen (28) is updated and the nurse or medical practitioner is advised to proceed. The nurse or medical practitioner draws the medicine into the syringe and puts the syringe between the guides on the transparent tray of the apparatus (21). The apparatus (21) decides if the amount of medicine in the syringe is correct and safe, and if so, prints a label (23). The apparatus (21) updates a patient database with all pertinent information. The nurse or medical practitioner puts the label on the syringe.

FIG. 5 shows an embodiment of a flowchart delineating the operation of the software of the apparatus (21). Said embodiment delineates a broad concept of the operation of the software of the apparatus (21). Said flowchart starts with the nurse or medical practitioner accomplishing a successful login (75) using the graphics user interface or GUI (32) on the touchscreen (28). The nurse or medical practitioner enters (76) the current patient using the graphics user interface or GUI (32) on the touchscreen (28). In some embodiments, the apparatus (21) is in communication with a patient database or Electronic Health Record of the patient and the software can receive (77) all pertinent data on the patient and can display pertinent data using GUI (32). The pertinent data can optionally include one or more medication orders pertaining to the patient, one or more medical conditions of the patient, information relevant to one or more medical conditions of the patient or any combination of the foregoing. All pertinent data of the patient, for example the patient's EHR, can be stored in the memory of the apparatus (21), stored in memory remote of the apparatus (21), for example offsite of the apparatus (21), or stored in any combination of the foregoing. In some embodiments, the nurse or medical practitioner manually enters (77) the pertinent data of said current patient using GUI (32). The nurse or medical practitioner picks (78) the type of drug or medicine using GUI (32). The nurse or medical practitioner enters (78) the dosage of said drug or said medicine using GUI (32). The nurse or medical practitioner holds a vial of medicine (39) below the camera (34), the camera takes a picture of the label (79) of said vial (39) of medicine, and the software analyzes (79) the image of the barcode on the label (40) of said vial (39) of medicine, and the software decides (79) if said vial of medicine is the correct medicine. If the vial (39) of medicine is not correct, the GUI (32) asks the nurse or medical practitioner to redo the process of picking the medicine (78). If the vial (39) of medicine is correct, the GUI (32) asks the nurse or medical practitioner to draw (80) the medicine into a syringe, and the GUI (32) asks the nurse or medical practitioner to tap the syringe to eliminate air bubbles (81), and the GUI (32) asks the nurse or medical practitioner to place (82) the cap (43) or first end on to the syringe (30) or container. GUI (32) asks the nurse or medical practitioner to place (82) the syringe (30), or container having first and second ends, on top of the transparent tray (25). The nurse or medical practitioner pushes a button on GUI (32) to alert the software that the syringe (30) is ready for analysis (83). The camera (34) takes a picture (84) of the syringe (30). The software scans (85) said picture and the software analyzes the data to determine (85) if the syringe is the correct size, to determine (85) the volume of liquid, and to determine (85) the presence of air or air bubbles present in the syringe (30). If the size of the syringe (30) is not correct (80), the software goes back to step (78) and the GUI (32) asks the nurse or medical practitioner to pick the medicine or drug and dose (78). If the size of the syringe (30) is correct (80), the software decides if the volume of medicine or the volume of drug is correct (86). and the GUI (32) asks the nurse or medical practitioner to pick the medicine or drug and dose (78). If the volume of medicine or the volume of drug is not correct, the software decides if there is too much volume (89) or too little volume (88). If there is too little volume (88) then GUI (32) takes the nurse or medical practitioner back to draw up more medicine (80) into the syringe (30). If there is too much volume (88) then GUI (32) asks the nurse or medical practitioner to squirt out an amount of the medicine which can allow for proper volume to be achieved, then GUI (32) asks the nurse or medical practitioner to recap the syringe (82). If the volume is correct (87) then the software analyzes the picture to determine if there is air or air bubbles (90) in the syringe (30). If there is a harmful amount of air detected by the software, the GUI (32) asks the nurse or medical practitioner to tap the syringe to eliminate air bubbles (81). If there is not a harmful amount of air detected by the software, the GUI (32) alerts the nurse or medical practitioner of success (91). The software updates the electronic health record EHR if an EHR is available (92). The software tells the printer (22) to print (93) a label (23) for the nurse or medical practitioner to affix to the syringe (30) or container having first and second ends. It is appreciated that steps identified in FIG. 5, or described herein with respect thereto, can be eliminated or changed, or further steps added, and be within the scope of the present invention, and other aspects of the invention described herein may or may not incorporate some or all of the steps identified in FIG. 5 or described herein with respect thereto.

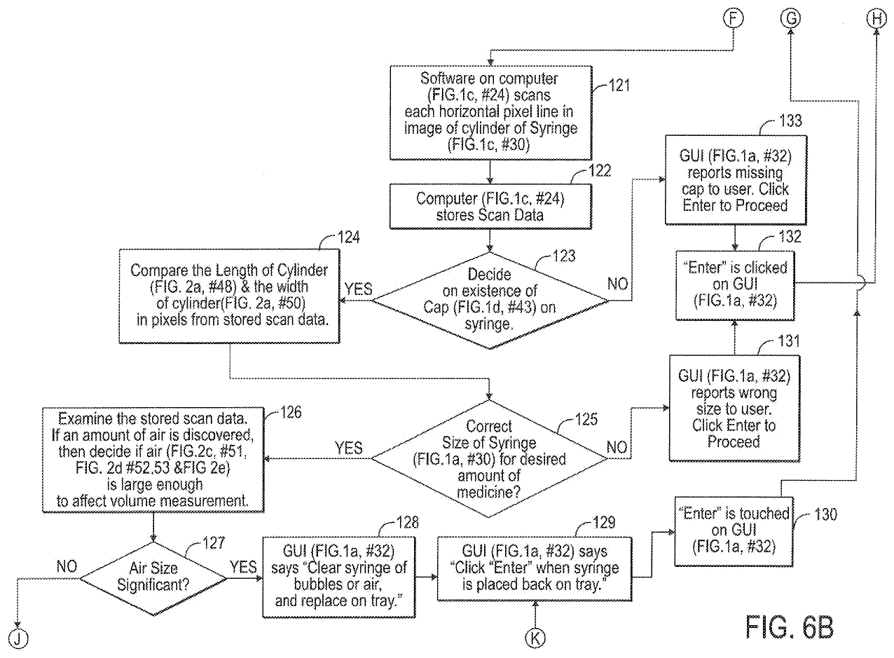

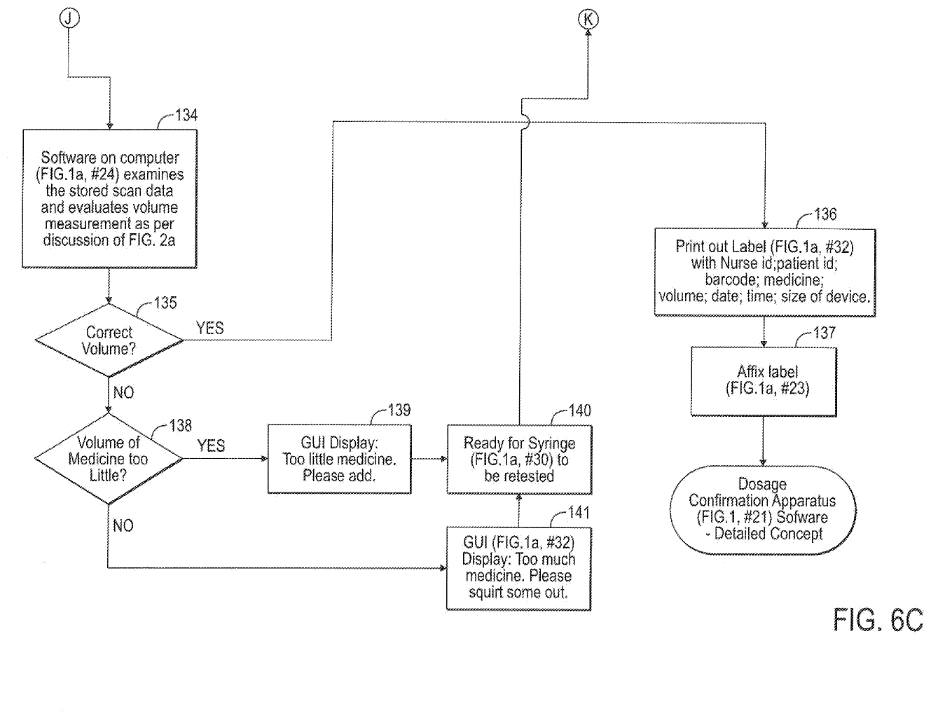

FIG. 6a-6c show an embodiment of a flowchart delineating the operation of the software of the apparatus (21). Said embodiment delineates a more detailed concept of the operation of the software of the apparatus (21) than FIG. 5. Said flowchart starts (FIG. 6a) with the nurse or medical practitioner tapping the graphics user interface or GUI (32) on the touchscreen (28) to wake up the sleeping computer (24) if needed (97). The GUI (32) on the touchscreen (28) displays the login screen (98). The nurse or medical practitioner enters username and password, then presses the “Enter” button (99). Alternatively, the nurse or medical practitioner scans their Identification Badge, enters password, then presses the “Enter” button (99). The software decides as to whether the login is authentic (100). If the login is not authentic, an alert states that “Login Failed”, and the nurse or medical practitioner can revisit step (99) and try to login again. If the login is authentic, the nurse or medical practitioner selects (102) the current patient using the graphics user interface or GUI (32) on the touchscreen (28). The software determines if the patient identifier is recognized as valid (103). If the software determines that the patient identifier is not valid, the graphics user interface or GUI (32) displays an alert to try again (104) and step (102) is repeated. If the software determines that the patient identifier is valid, then the software extracts pertinent patient information from the patient database (105) or, if said patient information is not available, asks the nurse or medical practitioner to enter the information (105) in the graphics user interface or GUI (32) on the touchscreen (28). Said information may include, but is not limited to, type of medicine, one or more medication orders, dosage or range of doses of medicine required, blood glucose level, medical conditions of the patient, information pertaining to medical conditions of the patient or any combination of the foregoing. Said information is displayed (106) on the graphics user interface or GUI (32) on the touchscreen (28). The graphics user interface or GUI (32) tells the nurse or medical practitioner to position a vial of medicine below the camera (34) on the transparent tray (25) between guides (27) with the barcode on the label facing upwards (107). The nurse or medical practitioner presses the “Enter” button (108) in the graphics user interface or GUI (32) on the touchscreen (28) when the vial is in position. Software on computer (24) instructs (109) camera (34) to read barcode (40). Software compares information (109) on barcode (40) to information the software already has acquired of the patient, and software analyzes if the medicine is the correct type of medicine for the patient (110). If the vial of medicine is not correct, the GUI (32) alerts the nurse or medical practitioner with a message “Wrong medicine or misread” (112). GUI (32) asks the nurse or medical practitioner to redo the process of positioning correct medicine (107). If the vial of medicine is correct, the GUI (32) alerts the nurse or medical practitioner (111) “Medicine Correct. Please place filled syringe on tray between the guides, being sure that the wings or flanges of the syringe are on the tray.” The GUI (32) alerts the nurse or medical practitioner (119) to press “Enter” button when ready. When the nurse or medical practitioner presses the “Enter” button, the software instructs the camera (34) to take a picture (120).

FIG. 6b continues the flow of an embodiment of a flowchart delineating the operation of the software of the apparatus (21). Software scans each horizontal pixel line (121) in image of syringe (30) or container having first and second ends. Software stores scanned data (122) for analysis. Software decides (123) on the existence of a cap (43) or first end in place on the top of the syringe (30) or container. If the cap or first end is not in place, the GUI (32) reports to the nurse or medical practitioner that the cap or first end is missing and to touch “Enter” to proceed (133). The software sends the user back to step #111. If the cap or first end is in place, the software assesses the length in pixels of the cylinder (48) of the syringe (30), or container having first and second ends, and the width of the cylinder (48) of the syringe (30), comparing (124) the length and width of the cylinder (48) of the syringe (30), or container having first and second ends, to a library of known lengths and known widths of known sizes of syringes (30) or containers having first and second ends. The software determines (125) if the calculated size of the syringe, or container having first and second ends, is the proper size for the present dosage amount. If the calculated size of the syringe, or container having first and second ends, is not the proper size, GUI (32) reports the wrong size to the user. GUI (32) alerts the nurse or medical practitioner (119) to press “Enter” button when ready. The software sends the user back to step #111. If the calculated size of the syringe, or container having first and second ends, is the proper size, the software can analyze the stored scan data and determine (126) if there is an amount of air discovered which is significant to the current application of the dose. If there is said amount of air, then GUI (32) says (128) “Clear syringe of bubbles or air, and replace on tray.” GUI (32) says (129) “Click Enter when syringe is placed back on tray.” When “Enter” is touched (130) on GUI (32), the software restarts at 120 by taking another picture. If there is not a significant amount of air found in step 127, FIG. 6c shows that the software examines the stored scan data and evaluates (134) volume measurement of medicine in the syringe (30). If the volume of medicine (135) is not correct, then the software must decide if the volume of medicine is too little or too much. If the volume is too little (138) GUI (32) displays (139) “Too little medicine. Please add”. If the volume is too much GUI (32) displays (141) “Too much medicine. Please squirt some out.” The software is alerted when the syringe, or container having first and second ends, is ready to be retested (140). The software then goes to step #129 and proceeds again from step #129. If the volume is correct the software prints out label (23) with pertinent information (136) such as nurse id or medical practitioner id, patient id, medicine, volume, date, time, size of device. The nurse or medical practitioner affixes (137) the label to the syringe or container having first and second ends. It is appreciated that steps identified in FIG. 6, or described herein with respect thereto, can be eliminated or changed, or further steps added, and be within the scope of the present invention, and other aspects of the invention described herein may or may not incorporate some or all of the steps identified in FIG. 6 or described herein with respect thereto.

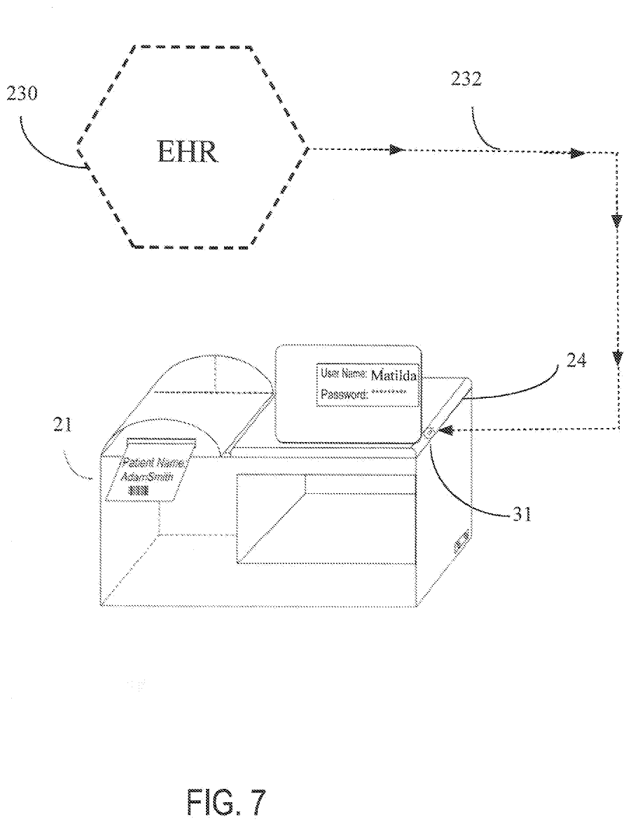

The method and apparatus of the invention can optionally access the medical conditions of the patient, which can include the patient's medical history, for use in determining a proper dosage of a medicament for the patient. The medical conditions include any characteristic, reading, measurement or fact pertaining to the patient, for example an insulin level of a patient, the blood pressure of a patient, a cholesterol level of a patient, a pulse of a patient, a blood or other fluid reading of a patient, a heart condition or reading of a patient, any cardiovascular reading, measurement or condition of a patient or any combination of the foregoing, a heart condition The medical conditions of the patient can optionally be obtained from health records of the patient, for example from the EHR of the patient. In this regard, for example, computer processor 24 can optionally receive a real-time feed of data 232 from the EHR 230 of the patient, for example through a suitable communications port 31 of the apparatus 21 (see FIG. 7). The data feed 232 can optionally include one or more medication orders 240 for the patient, and any suitable or relevant medical conditions of the patients, including for example laboratory results (LAB), patient demographics for example Admission, Discharge, and Transfer (ADT) or any combination of the foregoing. A medication order can optionally include a prescribed dosage of a medicament, a prescribed dosage or a range of prescribed dosages of a medicament as a function of one or more medical conditions of the patient, any correlation of dosages and medical conditions of a patient or any combination of the foregoing. The portal 232 can optionally be one-way, for example solely in the direction from the EHR to apparatus 21 in order to ensure that the apparatus 21 cannot write to the EHR 230 so as to change the EHR of the patient in any way. Portal feed 232 is can be easy to install in the field since either no processing or minimal processing is required of the owners of the EHR 230 to provide the feed 232.

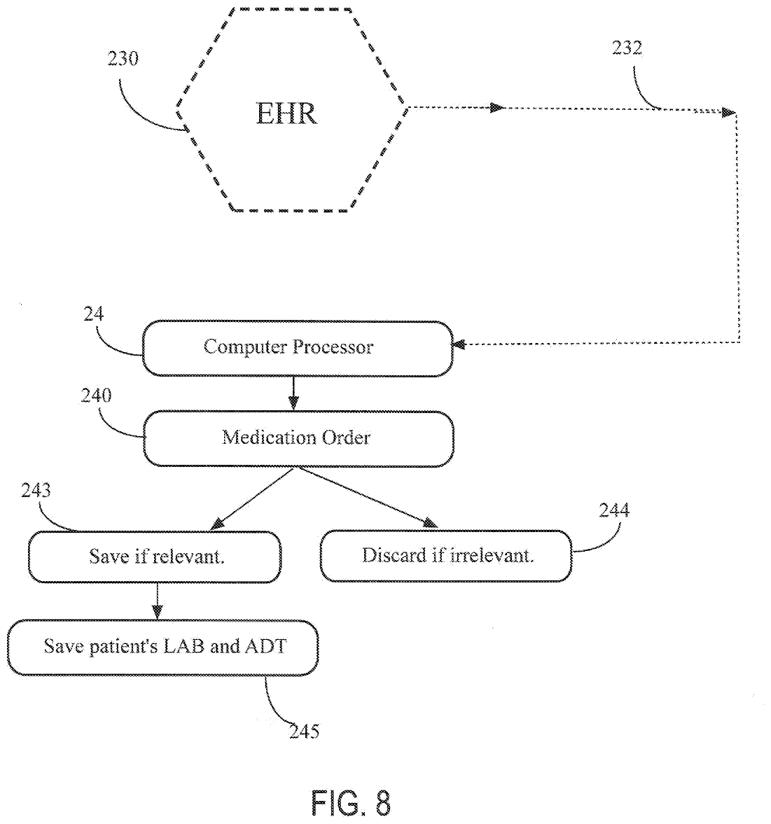

As shown in FIG. 8, for example, computer processor 24 of the apparatus 21 can examine the one or more medication orders 240 pertaining to the patient in a step, discard those medication orders that are not relevant, for example in step 244, and save those medication orders that are relevant, for example in step 243. Processor 24 can optionally save certain medical conditions of the patient, for example those associated with the patient referred to in the relevant medical order 240, in a suitable step such as step 245. The saved medical conditions can include, for example relevant LAB and ADT of the patient.

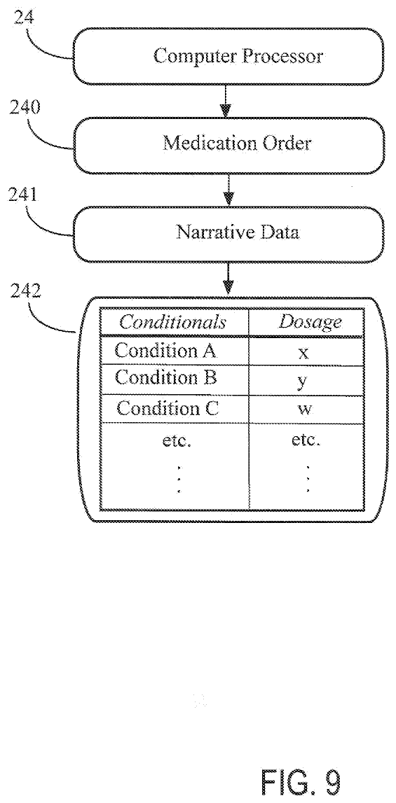

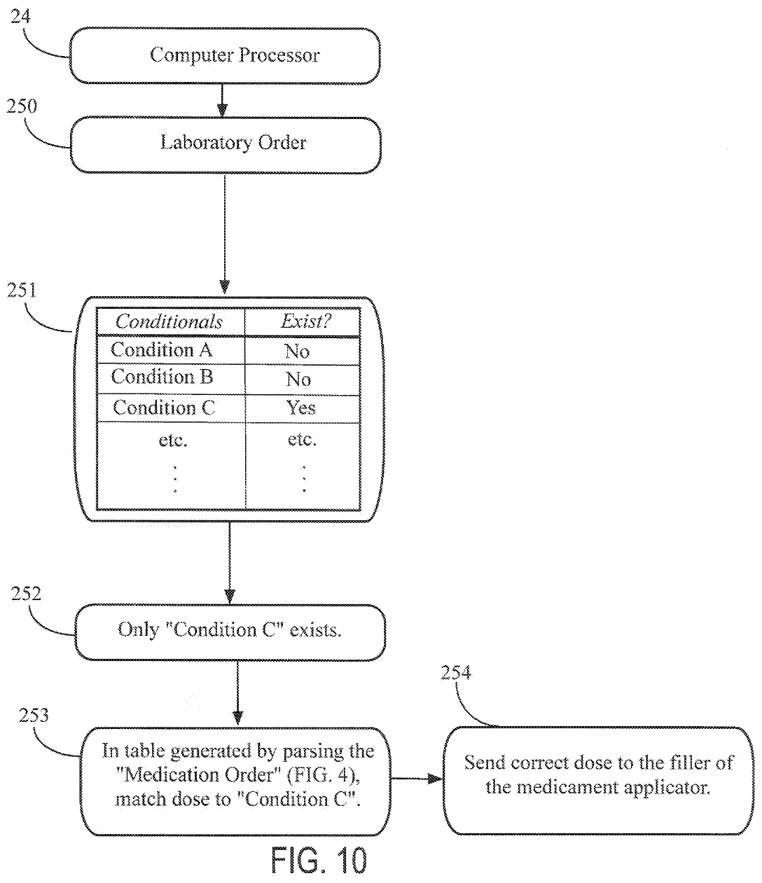

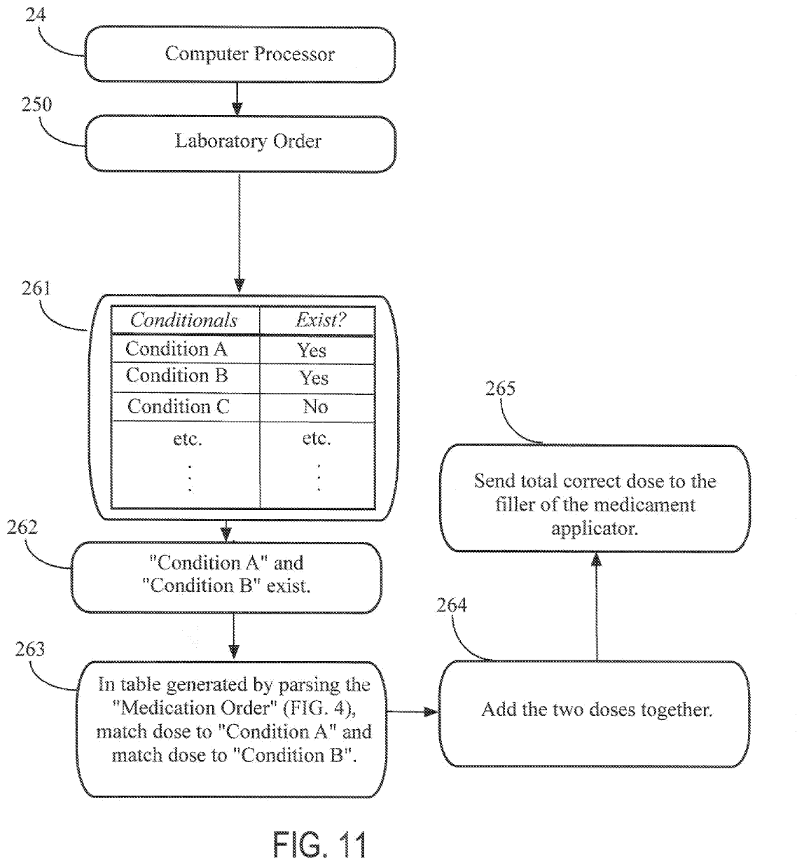

As part of any examination step, for example of the EHR of the patient, computer processor 24 can optionally examine the medication orders 240, for example a saved medication order from the method of FIG. 8 or otherwise, for any narrative data 241 included in the saved medication order pertaining to medication doses, correlations of doses to medical conditions of a patient or both (see FIG. 9). The narrative data 241, or portions thereof, can optionally be changed by processor 24 into discrete dosage data in any suitable manner, for example by any suitable character recognition, natural language processing or conversion technique. Each discrete dosage data can optionally be further changed, for example by processor 24 in any suitable step, to relate directly to certain logical conditions 242 that should optionally be present in order for a related discrete dosage to be added to, subtracted from or otherwise reflected in the total dosage needed to be provided to the patient. The conditions 242 can optionally be referred to as medical conditions of the patient. Some of all of the discrete dosage data can optionally be a function of one or more medical conditions of the patient, for example as evaluated by processor 24. In the sample case shown in FIG. 9, for example, the medication order 240 provides, for example by means of narrative data 241, that if medical condition A exists, the dosage is x; if medical condition B exists, the dosage is y; if medical condition C exists, the dosage is w, and possibly more. The discrete doses can optionally be referred to as conditional doses.

Using computer processing in place of human calculations to measure the conditional or discrete doses can increase the accuracy of complex conditional measurements and is therefore desirable. Increasing the processing speed of the measurement process by using computer processing to replace human calculations to measure the conditional doses, or discrete doses, can increase throughput and therefore save time, which can be desirable in the marketplace. To accomplish accurate and safe dosing of the patient, the reliability of a computer processing the measurement calculations and conditions of dosage before the administration of that dose to the patient can save lives and ease suffering from human calculation error.

FIG. 10 illustrates a sample process that may take place when certain medical conditions of the patient, for example laboratory records or LAB of the patient 250, are analyzed, for example in step 251 by processor 24 of apparatus 21, in order to see if certain conditions 242 contained in the medication order 240, for example in a saved medication order from the method of FIG. 8 or otherwise, exist that affect the determination of the dosage provided to the patient. FIG. 10 illustrates the case where the processor 24 determines that there is only one condition 252 from the medication order which exists in the laboratory records 250 of the patient. The condition 252 can be matched, for example in step 253 by the processor 24, with the discrete or conditional number or adjustment which is paired with the condition 252. This discrete or conditional number or adjustment, describing the amount of the discrete or condition dose or dose adjustment, can be calculated or factored into the aggregate dose sent to the filler of the medicament applicator, for example in step 254. For example, the discrete or conditional dose or dose adjustment may optionally be added to or subtracted from the base dose, for example the base dose found in the relevant medication order. The discrete or conditional dose or dose adjustment may optionally pertain to a range of measurements of a medical condition, and for example determine the appropriate dose within such a range of measurements of a medical condition. In the sample case shown in FIGS. 9-10, for example, in step 251 of FIG. 10 it is determined that Condition C exists, which in accordance with the medication order 240 and medical conditions 242 of FIG. 9 requires that the dosage be w. The filler can optionally be a suitable medical professional, for example a nurse, pharmacist, pharmacy technician, physician or any combination of the foregoing, an automated device or both. For example, an automated filler can optionally include a robotic arm. The medicament application can optionally be any of the containers disclosed herein.

FIG. 11 illustrates a sample process, similar to the process shown in FIG. 10, where certain medical conditions of the patient, for example laboratory records or LAB of the patient 250, are analyzed, for example in step 261 by processor 24 of apparatus 21, in order to see if certain conditions 242 contained the medication order 240, for example in a saved medication order from the method of FIG. 8 or otherwise, exist that require adjustment of or reflection in the dosage provided to the patient. In the sample process shown in FIG. 11, processor has determined from its analysis of certain medical conditions of the patient in laboratory records 250 that more than one condition 242 contained in the medication order 240 exists. In the sample process of FIG. 11, a plurality of conditions 262, for example two conditions 262, in the medication order exist. Each of the plurality of conditions, for example each of the two conditions 262, can be matched can be matched, for example in step 263 by the processor 24, with the discrete or conditional number or adjustment which is paired with the respective condition 262. These multiple discrete numbers or adjustments can be added together or otherwise combined by the processor 24, for example in step 246, to arrive at a total discrete or conditional dose or dose adjustment that can be calculated or factored into the aggregate dose sent to the filler of the medicament applicator, for example in step 265. For example, the total discrete or conditional dose or dose adjustment may optionally be added to or subtracted from the base dose, for example the base dose found in the relevant medication order. The total discrete or conditional dose or dose adjustment may optionally pertain to a range of range of measurements of a medical condition, and for example determine the appropriate dose within such a range of measurements of a medical condition. In the sample case shown in FIGS. 9 and 11, for example, in step 262 of FIG. 11 it is determined that from a review of the medical conditions 261 in laboratory order 250 that Condition A and Condition B both exist, which in accordance with the medication order 240 and medical conditions 242 of FIG. 9 requires that the dosage be the sum of x and y. Any suitable filler can be utilized, for example any of the fillers disclosed above.

The filler can obtain the medicament and can verify that the correct type of medicament is present in the manner, for example by any suitable apparatus such as apparatus 21. For example, the filler can place the medicament applicator, for example any container 30 disclosed herein, on tray 25 of apparatus 21, for example as shown in FIG. 1a, and the apparatus 21 can verify the accuracy of the type of medicament. Any suitable bar code technology can be used in such verification step, for example placed on the medicament applicator, disclosing the correct type of medicament and readable by apparatus 21.

The filler can fill a suitable medicament applicator, for example any container 30 disclosed herein, to the correct dose, and verify that the correct dose has been filled in any suitable manner, for example in any manner disclosed herein. For example, the filler can place the medicament applicator 30 on the measurement area 25 and the apparatus 21 can verify the accuracy of the measurement of the dosage. The apparatus can optionally use computer vision or any technique disclosed herein to verify the accuracy of the dose. If the type of medicament is verified to be correct and the dosage of the medicament in the applicator has been verified to be correct, then a barcode can optionally be issued, for example on label 23, that contains the dose mandated by the EHR. The barcode can then optionally be applied to the applicator to identify that the applicator contains the correct type and dose for that patient. The barcode of the dose and type of medicament, optionally as well as the patient information, can optionally be in a form that the EHR normally understands when a dosage barcode is read by the EHR.

After the applicator, for example container 30, has been labeled with the dose and the type of medicament, the applicator can optionally be scanned along with the patient identification barcode before the administration of the medicament to the patient. The EHR can optionally record the administration of the dose and type.

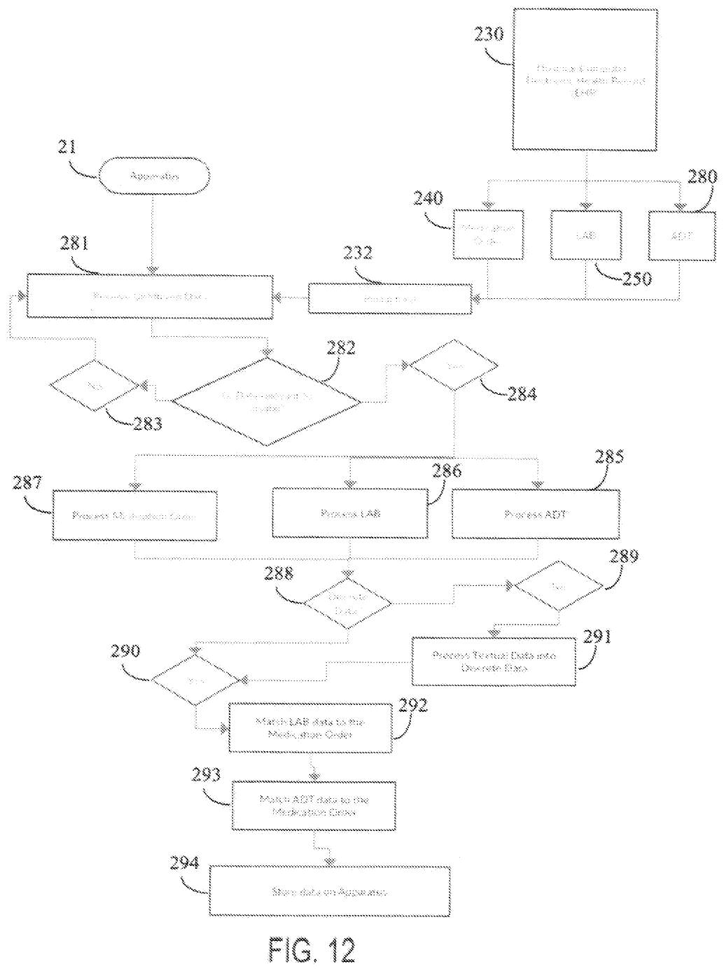

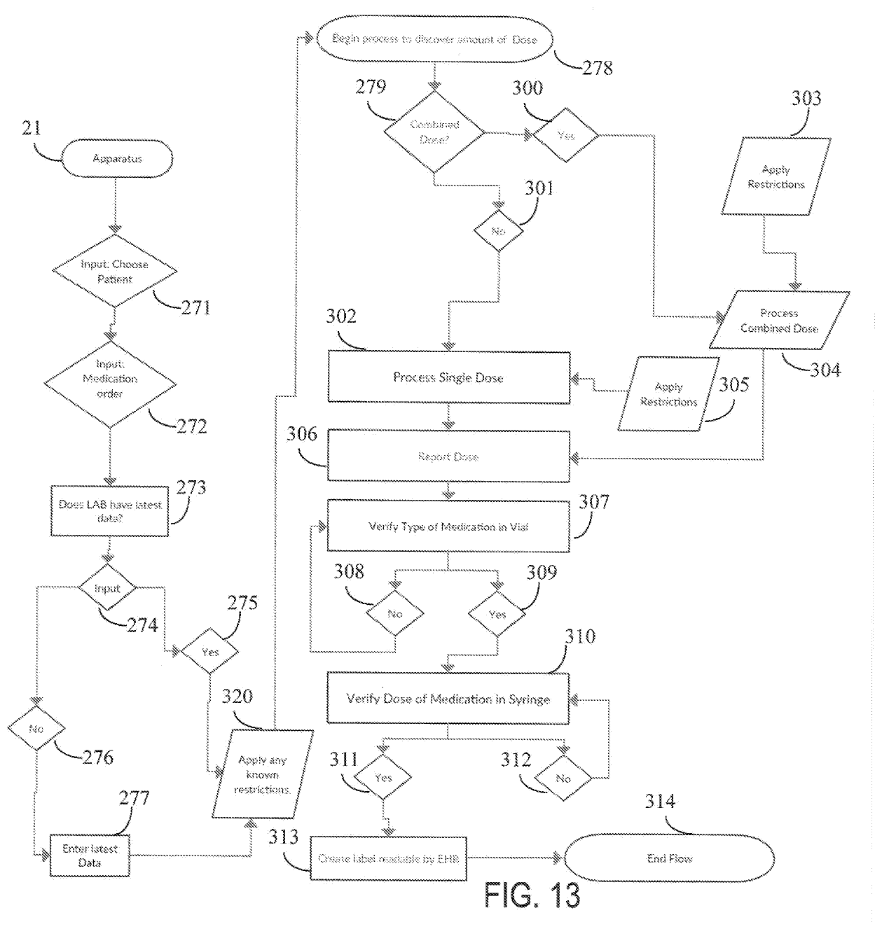

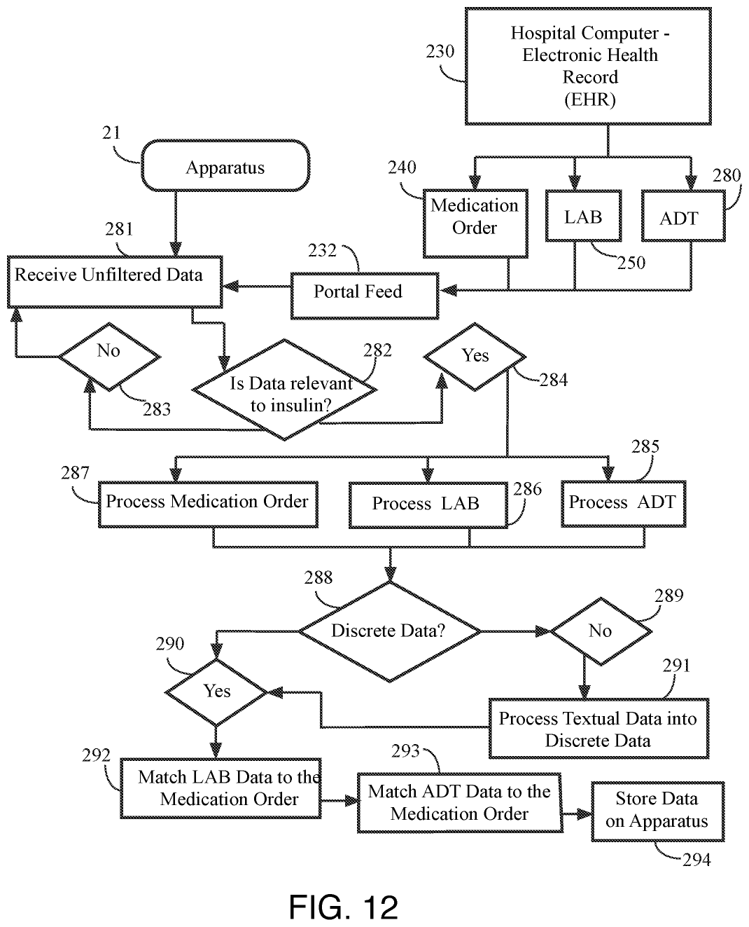

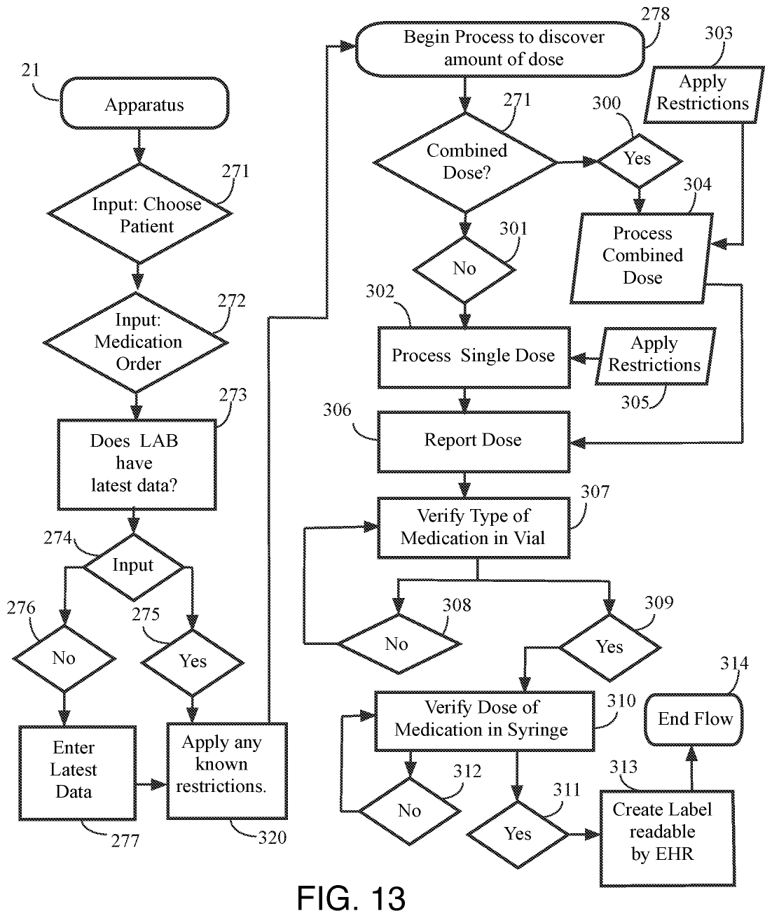

As discussed above, apparatus 21 can be designed or configured to receive information from an EHR 230 of a patient. A flowchart of a method and apparatus of the invention for analyzing the EHR, for example to determine a proper dosage of a medicament in accordance with a medication order, is set forth in FIGS. 12 and 13. Although the flow chart references insulin as the medicament, the flow chart is applicable to any medicament. The information obtained by apparatus 21 from the EHR can optionally include patient medication orders 240. Such information can optionally additionally include lab results or LAB 250 of the patient, for example referred to in or applicable to the medication orders 240. The information can optionally additionally include information on the patient's demographics or ADT 280. The information can optionally additionally include any other medical or other conditions of the patient relevant to determining a proper dosage of a medicament for the patient. The information from EHR 230, which can optionally be referred to as unfiltered data 281, can be received by the apparatus in any suitable manner, for example a suitable portal feed such as portal feed 232. Apparatus 21 can optionally be configured to cull or analyze the unfiltered information 281 for specific information that contain reference to insulin, or another medicament, for example in step 282. If unfiltered information 281 is not relevant to insulin, processor 24 optionally ceases analyzing it, for example in step 283. If unfiltered information 281 is relevant to insulin, the processor 24 is directed to further analyze such filtered information, for example in step 284.

Relevant medication orders in the filtered information can be processed for analyzed by processor, for example in step 287 of FIG. 12, relevant LAB information in the filtered information can be processed or analyzed by processor 24, for example in step 286 and relevant ADT information in the filtered information can be processed or analyzed by processor, for example in step 285. Other medical or other conditions of the patient in the filtered information can likewise be processed or analyzed by processor 24. The dose and the type can be either independent of laboratory values or corresponding to particular laboratory values. The dose and the type can optionally be dependent on medical conditions of the patient, which for example can be stated textually or numerically. The medical conditions can optionally be contained in the LAB information 250, the ADT information 280 or both. The LAB and/or the ADT can optionally contain textual descriptions of said current conditions or numbers referring to said current conditions, or both. In this regard, the filtered information can optionally be further analyzed by processor 24 to determine whether it contains any discrete data such as a numeric description of the required base insulin dose, any conditional insulin dose or any range of insulin doses as a function of one or more medical conditions of the patient, for example in step 288. If so, the processor obtains such discrete data for further processing or analysis, for example in step 290. If not, the processor can optionally direct the filter information to be further processed, for example in step 289. Such further processing can optionally include processing any textual data contained in the filtered information, for example in step 291, for any textual description of insulin base dose, conditional dose required or any range of insulin doses as a function of one or more medical conditions of the patient and insulin type required, for example by utilizing any suitable optical character recognition technology. The medical conditions can optionally include blood glucose levels of the patient. In addition to the foregoing, medical conditions or numbers can optionally be inputted into the apparatus, for example by a health care practitioner. The computer processor 24 of apparatus 21 can optionally match or correlate filtered LAB 250 data to filtered medication orders 240, for example in step 292. The computer processor can optionally match or correlate filtered ADT 280 data to filtered medication orders 240, for example in step 293. The outputs from such match or correlations steps can optionally be sent to the memory of apparatus 21 and thus stored on the apparatus 21.

The apparatus can optionally calculate the amount of the dose prescribed in each particular case, for example as set forth in FIG. 13. The apparatus 21 takes input, for example from the user of the apparatus, as to what patient is to be treated, for example in step 271. The apparatus provides all relevant medication orders for the patient to the user, which can be the filler of the order. The filler chooses which medication order or orders to process, for example in step 272. The apparatus 21 can provide the latest LAB data of the patient to the filler, and the filler decides if there is any more current LAB data, for example in step 273, and if so, for example in step 276, the filler inputs said more current data into the apparatus, for example in step 277. The apparatus 21 takes input so as to determine the existence or absence of any additional doses needed, for example in step 279, incorporates any said existent additional doses from other additional dosage orders, for example in step 304, within the same medical order or from other orders within other medication orders, for example in step 304, provides a discrete number instruction to the filler of the insulin dose, for example in step 306, or any combination of the foregoing. The filler fills the dose into the medicament applicator, for example a syringe. The apparatus can optionally verify the type of medicament for the container, for example in step 307, and confirm in step 309 or deny in step 308 that the type of medication to be filled in the container is correct. The apparatus can optionally verify the filled dose in the syringe or container, for example in step 310, and confirm in step 311 or deny in step 312 that the dose is correct. If processor determines that the dose is confirmed as correct, the apparatus can optionally create a barcode label, for example in step 313, to apply to the syringe or other medicament container. The barcode label can optionally contain the confirmed dose, patient information, the type of medicament or any combination of the foregoing. The barcode label can optionally be readable by an Electronic Health Record system.

The medicament applicator can optionally be a pen type applicator. The apparatus can be used with another medicament instead of insulin. The communication access by the apparatus with the EHR can optionally be wireless or wired. The data communicated by the EHR to the apparatus can optionally be the total data of the EHR or a portion of the total data of the electronic health record, minimally including the medication order or medication orders of each patient, the demographics such as ADT of the patient, and the lab results such as LAB of the patient.

In some embodiments, the processor is optionally configured to verify, at a later time when any manually inputted manual data was inputted into said apparatus, that the medical conditions inputted manually coincide with the corresponding data in the EHR when said corresponding data becomes available in the EHR. Said embodiment would alert administers of the EHR if the data does not coincide.

In some embodiments, an apparatus for use with computer memory having stored characteristics of a plurality of known containers to determine the volume of a liquid in one of the plurality of known containers having first and second ends for defining the volume of liquid is provided and includes a digital camera adapted for viewing the container, a processor electrically coupled to the camera and configured to optically detect certain characteristics of the container viewed by the camera and to access the computer memory having stored characteristics of the plurality of known containers, the processor being configured to compare the detected certain characteristics with the stored characteristics to identify the container from the plurality of known containers, the processor being configured to calculate the volume of the container as a function of the distance between the first and second ends of the container as viewed by the camera.

The apparatus can include an input interface for receiving the identity of the liquid and a printer, the processor being configured to cause the printer to print a label which includes the identity of the liquid. The processor can be configured to determine whether the container is filled entirely with the liquid. The apparatus can be configured to provide a first output signal if the container is entirely filled with the liquid and a second output signal if the container is not entirely filled with the liquid. The apparatus can include a light assembly for projecting first and second colors of light through the container, wherein the processor is configured to determine whether the container is filled entirely with the liquid by analyzing a pattern of the first and second colors emitted from the container and viewed by the camera. The processor can be configured to determine whether the container is filled entirely with air by analyzing a pattern of the first and second colors emitted from the container and viewed by the camera. The processor can be configured to determine whether the container contains bubbles of air by analyzing a pattern of the first and second colors emitted from the container and viewed by the camera. The apparatus can include a structure having a support for receiving the container, wherein the light assembly is carried by the structure and the camera is carried by the structure opposite the light assembly relative to the container. The light assembly can include a light source and a first filter of the first color and a second filter of the second color disposed alongside the first filter so that a first portion of the light from the light source travels through the first filter and a second portion of the light from the light source travels through the second filter. The pattern of the first and second colors can include the first color disposed alongside the second color. The container can be a syringe having a barrel and a plunger, the barrel having a first end provided with a fluid exit port and a second provided with an opening and the plunger having an end for extending through the opening in the barrel for slidable disposition in the barrel, the first end of the container being the first end of the barrel and the second end of the container being the end of the plunger. The apparatus can include a printer, the processor being configured to cause the printer to print a label which includes the dose of the liquid.

In some embodiments, an apparatus for use in determining the presence of any air pockets in a volume of a liquid in a container is provided and includes a digital camera adapted for viewing the liquid within the container and providing at least one image of the liquid within the container and a processor electrically coupled to the camera for receiving the at least one image from the camera and configured to determine whether the liquid contains any air pockets based on the at least one image.

The apparatus can be configured to provide a first output signal if the liquid does not contain any air pockets and a second output signal if the liquid does contain any air pockets. The apparatus can include an input interface for receiving the identity of the liquid and a printer, the processor being configured to cause the printer to print a label which includes the identity of the liquid. The processor can be configured to print the label only if the liquid does not contain any air pockets. The apparatus can include a light assembly for projecting first and second colors of light through the container, wherein the processor is configured to determine whether the liquid contains any air pockets by analyzing a pattern of the first and second colors emitted from the container and viewed by the camera. The apparatus can include a structure having a support for receiving the container, wherein the light assembly is carried by the structure and the camera is carried by the structure opposite the light assembly relative to the container. The light assembly can include a light source and a first filter of the first color and a second filter of the second color disposed alongside the first filter so that a first portion of the light from the light source travels through the first filter and a second portion of the light from the light source travels through the second filter. The pattern of the first and second colors can include the first color disposed alongside the second color. The apparatus can include a printer, the processor being configured to cause the printer to print a label which includes the dose of the liquid.

In some embodiments, a method for use by an apparatus having a camera and a processor electrically coupled to the camera to confirm the dosage of a medicament in a container is provided and includes accessing the proper volume of the dosage from computer memory, viewing the container with the camera to obtain at least one image of the container, delivering the at least one image of the container to the processor, calculating the volume of the liquid in the container utilizing the at least one image and comparing the calculated volume to the proper volume.

The computer memory can be remote from the apparatus. The proper volume can be included in a patient's electronic health record stored in the computer memory remote from the apparatus. The computer memory can be included in the apparatus and electrically coupled to the processor.

In some embodiments, an apparatus for use with a hospital's electronic health records is provided and includes a processor configured to receive all real time information produced by said hospital's electronic health records, the processor configured to search and analyze the electronic health records for medication orders pertaining to an injectable medicament of interest, the processor configured to select all patient medication orders which contain reference to injectable medications of interest, the selected medical order containing a textual description of medication dose required and medication type required, the processor configured to select each of the medication order's related lab reports and patient demographics, the textual description prescribing various medication doses and medication types required contingent on laboratory, physiologic, or demographic parameters, the processor configured to analyze said medication order and said contingent information to calculate the amount of a prescribed medicament dose in said particular case, and the processor configured to provide the correct amount of dose as a discrete number instruction to the filler.

The apparatus can verify that the dose filled within the syringe corresponds to the physician order for that patient at that time and for the current conditions.

In some embodiments, an apparatus for use with electronic health records of a patient to provide a medicament dose instruction of a prescription for the patient is provided and includes a processor configured to search and analyze the electronic health records for a medication order pertaining to medicament, the processor configured to analyze the medication order for a prescribed medicament dose, the processor configured to analyze the electronic health records for relevant medical conditions of the patient referenced in the medication order, the processor configured to adjust the prescribed medicament dose as a function of the relevant medical conditions of the patient to arrive at an adjusted medicament dose, and the processor configured to output the adjusted medicament dose for filling the prescription.

The medicament can be insulin. The medical conditions can include blood glucose levels of the patient. The electronic health records can include an Electronic Health Record of the patient. The electronic health records can include lab results of the patient and information on the patient's demographics. The medication order can include a textual description of the prescribed medicament dose. The medication order can include a numeric description of the prescribed medicament dose. The prescribed medicament dose can be selected from the group consisting of a medicament dose stated textually, a medicament dose stated numerically and a medicament dose stated both textually and numerically. The medical conditions can be selected from the group consisting of medical conditions stated textually, medical conditions stated numerically and medical conditions stated both textually and numerically. The apparatus can include an input device electronically coupled to the processor for permitting manual input of the medical conditions. The processor can be configured to cumulate adjustments to the prescribed medicament dose from a plurality of medication conditions. The apparatus can include an output device electronically coupled to the processor for outputting the adjusted medicament dose in the form of a discrete number instruction to a filler of the adjusted medicament dose. The output device can be configured to print a barcode label containing the adjusted medicament dose. The apparatus can include an antenna electronically coupled to the processor for permitting wireless receipt of the electronic health records of the patient.

Several advantages may occur from the invention, for example with the use of an embodiment of apparatus (21). The amount of adverse medical events involving incorrect dosage can decrease and therefore can save lives and can ease suffering. Time can be saved by the apparatus' (21) processing taking less time than the time needed to locate a second nurse and having said second nurse check the accuracy of the first nurse's work. Accurate patient specific barcode for each dose can be provided. Links between diet and dose and blood glucose level, or other medical conditions of the patient, may be able to be analyzed due to the apparatus recording the data of each filling of syringes (30) or containers having first and second ends. Economic savings can occur due to the number of insulin pens decreasing. More effective treatment of said patient after release from the hospital can be achieved by the apparatus' 21 recording of more accurately confirmed past dosages administered of insulin and analyzing said dosages' relationship to blood glucose levels. The open design can allow for easier cleaning, and therefore thorough sanitation. The artificial intelligence invented for the apparatus (21) that allows a machine to easily and inexpensively tell the difference between air and clear liquid in a cylinder is a large advance in robotic vision. Other advantages of one or more aspects can be apparent from a consideration of the drawings and the drawings' descriptions.

What is claimed is:

1. An apparatus for use with electronic health records of a patient to provide a medicament dose instruction for the patient, comprising a processor, the processor configured to search the electronic health records for a medication order of the patient pertaining to the medicament, the medication order including dosage instructions correlated to at least one medical condition of the patient, the processor configured to search the electronic health records for the at least one medical condition of the patient referenced in the medication order, the processor configured to determine the prescribed medicament dose as a function of the at least one medical condition of the patient and output the prescribed medicament dose for filling.

2. The apparatus of claim 1, wherein the medicament is insulin.

3. The apparatus of claim 2, wherein the at least one medical condition includes blood glucose levels of the patient.

4. The apparatus of claim 1, wherein the electronic health records include an Electronic Health Record of the patient.

5. The apparatus of claim 1, wherein the electronic health records include lab results of the patient and information on the patient's demographics.

6. The apparatus of claim 1, wherein the medication order includes a textual description of the dosage instructions and the processor is configured to analyze the textural description for determining the dosage instructions.

7. The apparatus of claim 1, wherein the medication order includes a numeric description of the dosage instructions.

8. The apparatus of claim 1, wherein the dosage instruction is selected from the group consisting of a dosage instruction stated textually, a dosage instruction stated numerically and a dosage instruction stated both textually and numerically.

9. The apparatus of claim 1, wherein the at least one medical condition is selected from the group consisting of at least one medical condition stated textually, at least one medical condition stated numerically and at least one medical condition stated both textually and numerically.

10. The apparatus of claim 1, wherein the processor is configured to cumulate prescribed medicament doses derived from a plurality of medication order.

11. The apparatus of claim 1, further comprising an output device electronically coupled to the processor for outputting the prescribed medicament dose in the form of a discrete number instruction to a filler of the prescribed medicament dose.

12. The apparatus of claim 11, wherein the output device is configured to print a barcode label containing the prescribed medicament dose.

13. The apparatus of claim 1, further comprising an antenna electronically coupled to the processor for permitting wireless receipt of the electronic health records of the patient.

14. The apparatus of claim 1, further a sensor electrically coupled to the processor for verifying that a container is filled with the prescribed medicament dose.
